(19) United States
(12) Patent Application Publication
(10) Pub. No.: US 2004/0063730 A1
(43) Pub. Date: Apr. 1, 2004

(54) Pharmacuetical formulation comprising puyrazolo[4,-3-d]pyrimidines and antithrombotics, calcium antagonists, or prostaglandins or prostaglandin derivatives

(76) Inventors: Hans-Michael Eggenweiler, Darmstadt (DE); Volker Eiermann, Rodermark (DE)

Correspondence Address: Millen White, Zelano & Branigan, Arlington Courthouse Plaza I, 2200 Clarendon Boulevard Suite 1400, Arlington, VA, 22201, US

(21) Appl. No.: 10/451,105

(22) PCT Filed: Nov. 28, 2001
(86) PCT No.: PCT/EP01/13916

(30) Foreign Application Priority Data
Dec. 19, 2000 (DE) 100 63 224.6
Dec. 21, 2000 (DE) 100 63 882.1
Dec. 23, 2000 (DE) 100 64 993.9

(51) Int. Cl. A61K 31/519

(52) U.S. Cl. 514/262.1

(57) ABSTRACT

The invention relates to a pharmaceutical preparation containing at least one compound of formula (I), wherein R1, R2, R3, R4 and X have the meanings as cited in claim No. 1, and to their physiologically safe salts and/or solvates, and containing; a) at least one antithrombotic agent or; b) at least one calcium antagonist or; c) at least on prostaglandin or prostaglandin derivative for producing a medicament used for treating angina, hypertension, pulmonary hypertension, congestive heart failure (CHF), chronic obstructive pulmonary disease (COPD), cor pulmonale, right ventricular failure, atherosclerosis, conditions of reduced patency of the heart vessels, peripheral vascular diseases, cerebrovascular accident, bronchitis, allergic asthma, chronic asthma, allergic rhinitis, glaucoma, irritable bowel syndrome, tumors, kidney failure, cirrhosis of the liver, and for treating female sexual dysfunctions.

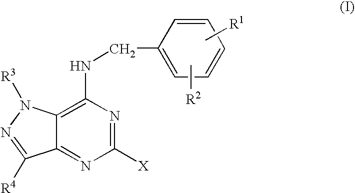

The invention relates to pharmaceutical formulations comprising at least one phosphodiesterase V inhibitor and/or physiologically acceptable salts and/or solvates thereof and at least one antithrombotic.

The invention relates in particular to pharmaceutical formulations comprising at least one compound of the formula I

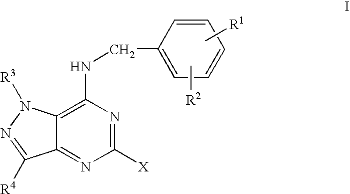

in which

R¹ and R² are each, independently of one another, H, A, OH, OA or Hal,

R¹ and R² together are alternatively alkylene having 3-5 carbon atoms, —O—CH₂—CH₂—, —CH₂—O—CH₂—, —O—CH₂—O— or —O—CH₂—CH₂—O—,

R³ and R⁴ are each, independently of one another, H or A,

X is R⁵, R⁶ or R⁷, each of which is monosubstituted by R⁸,

R⁵ is linear or branched alkylene having 1-10 carbon atoms, in which one or two CH₂ groups may be replaced by —CH═CH-groups, O, S or SO,

R⁶ is cycloalkyl or cycloalkylalkylene having 5-12 carbon atoms,

R⁷ is phenyl or phenylmethyl,

R⁸ is COOH, COOA, CONH₂, CONHA, CON(A)₂ or CN,

A is alkyl having from 1 to 6 carbon atoms, and

Hal is F, Cl, Br or I,

and/or physiologically acceptable salts and/or solvates thereof and

a) at least one antithrombotic or

b) at least one calcium antagonist or

c) at least one prostaglandin or prostaglandin derivative.

The invention furthermore relates to the use of the formulation for the preparation of a medicament for the treatment of angina, high blood pressure, pulmonary hypertension, congestive heart failure (CHF), chronic obstructive pulmonary disease (COPD), cor pulmonale, dextrocardiac insufficiency, atherosclerosis, conditions of reduced patency of heart vessels, peripheral vascular diseases, strokes, bronchitis, allergic asthma, chronic asthma, allergic rhinitis, glaucoma, irritable bowel syndrome, tumours, renal insufficiency, liver cirrhosis and for the treatment of female sexual disorders.

Pharmaceutical formulations consisting of other phosphodiesterase V (PDE V) inhibitors together with a second active ingredient are described in WO 00/15639.

Pyrimidine derivatives are disclosed, for example, in EP 201.188 and WO 93/06104.

The use of other PDE-V inhibitors is described, for example, in WO 94/28902.

Pharmaceutical formulations consisting of other phosphodiesterase V (PDE V) inhibitors together with calcium antagonists (=calcium channel blockers) are described in WO 00/15639.

Pharmaceutical formulations consisting of other phosphodiesterase V (PDE V) inhibitors together with a prostaglandin or prostaglandin derivative are described in WO 00/15639 and WO 0015228.

The use of (other) phosphodiesterase IV or V inhibitors in combination with a prostaglandin or prostaglandin derivative for the local treatment of erectile dysfunction is described in WO 9921558.

R. T. Schermuly et al. in the American Journal of Respiratory and Critical Care Medicine, 160, 1500-6 (1999), describe the therapeutic potential of prostaglandin I₂ (PGI₂) in aerosol form with systemic PDE inhibitors, preferably dual-selective PDE III/IV inhibitors, in low doses for acute and chronic pulmonary hypertension.

In Pneumologie (54, Suppl. 1, S42, 2000), R. Schermuly et al. describe the influence of PDE-V inhibition on prostacyclin-induced vasorelaxation in experimental pulmonary hypertonia.

The invention had the object of providing novel medicaments in the form of pharmaceutical preparations which have better properties than known medicaments which can be used for the same purpose.

This object has been achieved by the discovery of the novel preparation.

The compounds of the formula I and their salts have very valuable pharmacological properties and are well tolerated. In particular, they exhibit specific inhibition of CGMP phosphodiesterase (PDE V).

Quinazolines having a cGMP phosphodiesterase-inhibiting activity are described, for example, in J. Med. Chem. 36, 3765 (1993) and ibid. 37, 2106 (1994).

The biological activity of the compounds of the formula I can be determined by methods as described, for example, in WO 93/06104.

The affinity of the compounds according to the invention for cGMP and cAMP phosphodiesterase is determined by measuring their IC₅₀ values (concentration of the inhibitor needed to achieve 50% inhibition of the enzyme activity).

The determinations can be carried out using enzymes isolated by known methods (for example W. J. Thompson et al., Biochem. 1971, 10, 311). The experiment can be carried out using a modified batch method of W. J. Thompson and M. M. Appleman (Biochem. 1979, 18, 5228).

The compounds are therefore suitable for the treatment of illnesses of the cardiovascular system, in particular cardiac insufficiency, and for the treatment and/or therapy of impotence (erectile dysfunction).

The use of substituted pyrazolopyrimidinones for the treatment of impotence is described, for example, in WO 94/28902.

The compounds are effective as inhibitors of phenylephrine-induced contractions in corpus cavernosum preparations of rabbits. This biological action can be demonstrated, for example, by the method described by F. Holmquist et al. in J. Urol., 150,1310-1315 (1993).

The inhibition of the contraction demonstrates the effectiveness of the compounds according to the invention for the therapy and/or treatment of impotence.

The efficacy of the pharmaceutical formulations according to the invention, in particular for the treatment of pulmonary hypertension, can be demonstrated, as described by E. Braunwald in Heart Disease 5^{th} edition, W B Saunders Company, 1997, Chapter 6: Cardiac Catheterisation, 177-200.

The compounds of the formula I can be employed as medicament active ingredients in human and veterinary medicine. They can furthermore be employed as intermediates for the preparation of further medicament active ingredients.

The compounds of the formula I according to claim 1 and their salts are prepared by a process which is characterised in that

a) a compound of the formula II

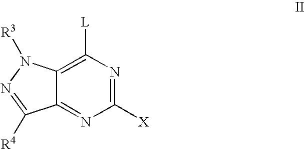

in which

R³, R⁴ and X are as defined above,

and L is Cl, Br, OH, SCH₃ or a reactive esterified OH group,

is reacted with a compound of the formula III

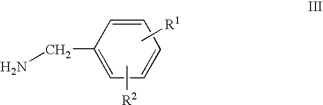

in which

R¹ and R² are as defined above, or

b) a radical X in a compound of the formula I is converted into another radical X by, for example, hydrolysing an ester group to a COOH group or converting a COOH group into an amide or into a cyano group,

and/or in that a compound of the formula I is converted into one of its salts.

The invention also relates to the use of all optically active forms (stereoisomers), the enantiomers, the racemates, the diastereomers, and the hydrates and solvates of the compounds.

The term solvates of the compounds of the formula I is taken to mean adductions of inert solvent molecules onto the compounds of the formula I which form owing to their mutual attractive force. Solvates are, for example, monohydrates or dihydrates or alkoxides.

Above and below, the radicals R¹, R², R³, R⁴, R⁵, R⁶, R⁷, R⁸, X and L are as defined under the formulae I, II and III, unless expressly stated otherwise.

A is alkyl having 1-6 carbon atoms.

In the above formulae, alkyl is preferably unbranched and has 1, 2, 3, 4, 5 or 6 carbon atoms and is preferably methyl, ethyl or propyl, furthermore preferably isopropyl, butyl, isobutyl, sec-butyl or tert-butyl, but also n-pentyl, neopentyl, isopentyl or hexyl.

X is an R⁵, R⁶ or R⁷ radical which is monosubstituted by R⁸.

R⁵ is a linear or branched alkylene radical having 1-10 carbon atoms, where the alkylene radical is preferably, for example, methylene, ethylene, propylene, isopropylene, butylene, isobutylene, sec-butylene, pentylene, 1- , 2- or 3-methyl butylene, 1,1- , 1,2- or 2,2-dimethylpropylene, 1-ethyl-propylene, hexylene, 1- , 2- , 3- or 4-methylpentylene, 1,1- , 1,2-, 1,3-, 2,2- , 2,3- or 3,3-dimethylbutylene, 1-or 2-ethylbutylene, 1-ethyl-1-methylpropylene, 1-ethyl-2-methylpropylene, 1,1,2- or 1,2,2-trimethylpropylene, linear or branched heptylene, octylene, nonylene or decylene.

R⁵ is furthermore, for example, but-2-enylene or hex-3-enylene.

A CH₂ group in R⁵ may preferably be replaced by oxygen.

Very particular preferance is given to ethylene, propylene, butylene or CH₂—O—CH₂.

R⁶ is cycloalkylalkylene having 5-12 carbon atoms, preferably, for example, cyclopentylmethylene, cyclohexylmethylene, cyclohexylethylene, cyclohexylpropylene or cyclohexylbutylene.

R⁶ is alternatively cycloalkyl, preferably having 5-7 carbon atoms.

Cycloalkyl is, for example, cyclopentyl, cyclohexyl or cycloheptyl.

Hal is preferably F, Cl or Br, but also I.

The radicals R¹ and R² may be identical or different and are preferably located in the 3- or 4-position of the phenyl ring. They are, for example, in each case independently of one another, H, alkyl, OH, F, Cl, Br or I or together are alkylene, such as, for example, propylene, butylene or pentylene, furthermore ethyleneoxy, methylenedioxy or ethylenedioxy. They are preferably also in each case alkoxy, such as, for example, methoxy, ethoxy or propoxy.

The radical R⁸ is preferably, for example, COOH, COOA, such as, for example, COOCH₃ or COOC₂H₅, CONH₂, CON(CH₃)₂, CONHCH₃ or CN, but in particular COOH or COOA.

For the entire invention, all radicals which occur more than once may be identical or different, i.e. are independent of one another.

The term antithrombotics also covers so-called anticoagulants and blood platelet aggregation inhibitors (thrombocyte aggregation inhibitors).

The invention relates in particular to pharmaceutical formulations comprising an antithrombotic, a calcium antagonist or a prostaglandin or prostaglandin derivative and at least one compound of the formula I in which at least one of the said radicals has one of the preferred meanings indicated above. Some preferred groups of compounds may be expressed by the following sub-formulae Ia to If, which conform to the formula I and in which the radicals not designated in greater detail are as defined under the formula I, but in which

in Ia X is R⁵, phenyl or phenylmethyl, each of which is substituted by COOH, COOA, CONH₂, CONA₂, CONHA or CN;

in Ib R¹ and R² together are alkylene having 3-5 carbon atoms, —O—CH₂—CH₂—, —O—CH₂—O— or —O—CH₂—CH₂—O—,

X is R⁵, phenyl or phenylmethyl, each of which is substituted by COOH, COOA, CONH₂, CONA₂, CONHA or CN;

in Ic R¹ and R² are each, independently of one another, H, A, OH, OA or Hal,

R¹ and R² together are alternatively alkylene having 3-5 carbon atoms, —O—CH₂—CH₂—, —O—CH₂—O— or —O—CH₂—CH₂—O—,

X is R⁵, phenyl or phenylmethyl, each of which is substituted by COOH, COOA, CONH₂, CONA₂, CONHA or CN;

in Id R¹ and R² are each, independently of one another, H, A, OH, OA or Hal,

R¹ and R² together are alternatively alkylene having 3-5 carbon atoms, —O—CH₂—CH₂—, —O—CH₂—O— or —O—CH₂—CH₂—O—,

X is alkylene having 2-5 carbon atoms, cyclohexyl, phenyl or phenylmethyl, each of which is mono-substituted by R⁸,

R³ is alkyl having 1-6 carbon atoms,

R⁴ is alkyl having 1-6 carbon atoms,

R⁸ is COOH or COOA,

A is alkyl having from 1 to 6 carbon atoms,

Hal is F, Cl, Br or I;

in Ie R¹ and R² are each, independently of one another, H, A, OH, OA or Hal,

R¹ and R² together are alternatively alkylene having 3-5 carbon atoms, —O—CH₂—CH₂—, —O—CH₂—O— or —O—CH₂—CH₂—O—,

R³ is alkyl having 1-6 carbon atoms,

R⁴ is alkyl having 1-6 carbon atoms,

X is —(CH₂)₂₋₅—R⁸, 4-R⁸-cyclohexyl, 4-R⁸-phenyl or 4-(R⁸-methyl)phenyl;

in If R¹ and R² are each, independently of one another, H, A, OH, OA or Hal,

R¹ and R² together are alternatively alkylene having 3-5 carbon atoms, —O—CH₂—CH₂—, —O—CH₂—O— or —O—CH₂—CH₂O—,

R³ is alkyl having 1-6 carbon atoms,

R⁴ is alkyl having 1-6 carbon atoms,

X is —(CH₂)₂₋₅—R⁸, in which one CH₂ group may be replaced by 0, or is 4-R⁸-cyclohexyl, 4-R⁸-phenyl or 4-(R⁸-methyl)phenyl,

R⁸ is COOH or COOA.

The invention preferably relates to a formulation comprising [7-(3-chloro-4-methoxybenzylamino)-1-methyl-3-propyl-1H-pyrazolo[4,3-d]pyrimidin-5-yl-methoxy]acetic acid and physiologically acceptable salts and/or solvates thereof and an antithrombotic.

Besides the free acid, the ethanolamine salt is preferred.

Preferred antithrombotics are vitamin K antagonists, heparin compounds, thrombocyte aggregation inhibitors, enzymes, factor Xa inhibitors, factor Vila inhibitors and other antithrombotic agents.

Preferred vitamin K antagonists are selected from the group consisting of dicoumarol, phenindione, warfarin, phenprocoumon, acenocoumarol, ethyl biscoumacetate, clorindione, diphenadione and tioclomarol.

Preferred heparin compounds are selected from the group consisting of heparin, antithrombin III, dalteparin, enoxaparin, nadroparin, parnaparin, reviparin, danaparoid, tinzaparin and sulodexide.

Preferred thrombocyte aggregation inhibitors are selected from the group consisting of ditazole, cloricromen, picotamide, clopidogrel, ticlopidine, acetylsalicylic acid, dipyridamole, calcium carbassalate, epoprostenol, indobufen, iloprost, abciximab, tirofiban, aloxiprin and intrifiban.

Preferred enzymes are selected from the group consisting of streptokinase, alteplase, anistreplase, urokinase, fibrinolysin, brinase, reteplase and saruplase.

Preferred antithrombotics are furthermore the blood platelet glycoprotein receptor (IIIb/IIIa) antagonists which inhibit blood platelet aggregation. Preferred compounds are described, for example, in EP 0 623 615 B1 on page 2 or in EP 0 741 133 A2, page 2, line 2, to page 4, line 56.

Preferred factor Xa and VIIa inhibitors are, for example,

a) the compounds of the formula I

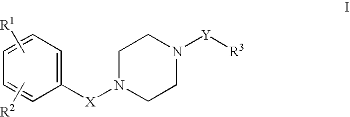

in which

R¹ is —C(═NH)—NH₂, which may also be monosubstituted by —COA, —CO-[C(R⁶)₂]ₙ—Ar, —COOA, —OH or by a conventional amino protecting group, or is

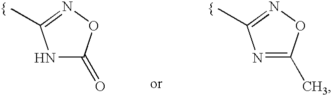

R² is H, A, OR⁶, N(R⁶)₂, NO₂, CN, Hal, NHCOA, NHCOAr, NHSO₂A, NHSO₂Ar, COOR⁶, CON(R⁶)₂, CONHAr, COR⁶, COAr, S(O)ₙA or S(O)ₙAr,

R³ is A, cycloalkyl, —[C(R⁶]Ar, —[C(R⁶)₂]ₙ—O—Ar, [C(R⁶)₂]ₙHet or —C(R⁶)₂═C(R⁶)₂—Ar,

R⁶ is H, A or benzyl,

X is absent or is —CO—, —C(R⁶)₂—, —C(R⁶)₂—C(R⁶)₂—, —C(R⁶)₂—CO—, —C(R⁶)₂—C(R⁶)₂—CO—, —C(R⁶)═C(R⁶)—CO—, NR⁶CO—, —N{[C(R⁶)₂]ₙ—COOR⁵}—CO— or —C(COOR⁶)R⁶—C(R⁶)₂—CO—,

Y is —C(R⁶)₂—, —SO₂—, —CO—, —COO— or —CONR⁶—,

A is alkyl having 1-20 carbon atoms, in which one or two CH₂ groups may be replaced by O or S atoms or by —CR⁶═CR⁶— groups and/or 1-7H atoms may be replaced by F,

Ar is phenyl or naphthyl, each of which is unsubstituted or monosubstituted, disubstituted or trisubstituted by A, Ar′, OR⁶, N(R⁶)₂, NO₂, CN, Hal, NHCOA, NHCOAr′, NHSO₂A, NHSO₂Ar′, COOR⁶, CON(R⁶)₂, CONHAr′, COR⁶, COAr′, S(O)NA or S(O)ₙAr,

Ar′ is phenyl or naphthyl, each of which is unsubstituted or monosubstituted, disubstituted or trisubstituted by A, OR⁶, N(R⁶)₂, NO₂, CN, Hal, NHCOA, COOR⁶, CON(R⁶)₂, COR⁶ or S(O)ₙA,

Het is a monocyclic or bicyclic, saturated or unsaturated heterocyclic ring system which contains one, two, three or four identical or different heteroatoms, such as nitrogen, oxygen and sulfur, and is unsubstituted or monosubstituted or polysubstituted by Hal, A, Ar′, COOR⁶, CN, N(R², NO₂, Ar—CONH—CH₂ and/or carbonyl oxygen,

Hal is F, Cl, Br or I,

n is 0, 1 or 2,

and salts thereof,

which are described in WO 9916751,

b) the compounds of the formula I

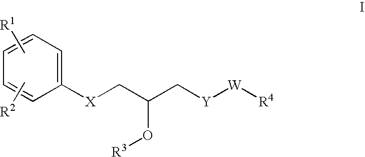

in which

R¹ is —C(═NH)—NH₂, which may also be monosubstituted by —COA, —CO—[C(R⁵)₂]ₘ—Ar, —COOA, —OH or by a conventional amino-protecting group, or is

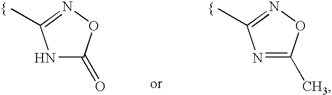

R² is H, A, OR⁵, N(R⁵)₂, NO₂, CN, Hal, NR⁵COA, NHCOAr, NHSO₂A, NHSO₂Ar, COOR⁵, CON(R⁵)₂, CONHAr, COR⁵, COAr, S(O)ₙRA or S(O)ₙAr,

R³ is R⁵ or —[C(R⁵)₂] COOR⁵,

R³ and X together are alternatively —CO—N—, with formation of a 5-membered ring,

where R³ is —C═O and X is N,

R⁴ is A, cycloalkyl, —[C(R⁵)₂]ₘAr, —[C(R⁵)₂]ₘHet or —CR⁵═CR⁵—Ar,

R⁵ is H, A or benzyl,

X is O, NR⁵ or CH₂,

Y is O, NR⁵, N[C(R⁵)₂]ₘ—Ar, N[C(R⁵)₂]ₘ-Het,

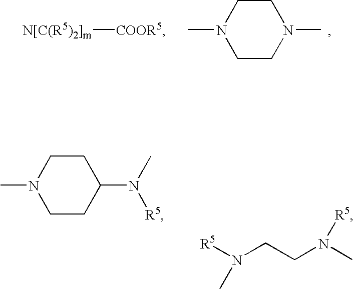

N[C(R⁵)₂]ₘ—CON(R⁵)₂, N[C(R⁵)_{z}]ₘ—CONR⁵Ar or N[C(R⁵)₂]ₘ—CONAr₂,

W is a bond, —SO₂—, —CO—, —COO— or —CONR⁵—,

A is alkyl having 1-20 carbon atoms, in which one or two CH₂ groups may be replaced by O or S atoms or by —CR⁵═CR⁵— groups and/or 1-7H atoms may be replaced by F,

Ar is phenyl or naphthyl, each of which is unsubstituted or monosubstituted, disubstituted or trisubstituted by R¹, A, Ar′, OR⁵, N(R⁵)₂, NO₂, CN, Hal, NHCOA, NHCOAr′, NHSO₂A, NHSO₂Ar′, COOR⁵, CON(R⁵)₂, CONHAr′, COR⁵, COAr′, S(O)ₙA or S(O)ₙAr,

Ar′ is phenyl or naphthyl, each of which is unsubstituted or monosubstituted, disubstituted or trisubstituted by R¹, A, OR⁵, N(R⁵)₂, NO₂, CN, Hal, NHCOA, COOR⁵, CON(R⁵)₂, COR⁵ or S(O)ₙA,

Het is a monocyclic or bicyclic, saturated or unsaturated heterocyclic ring system which contains one, two, three or four identical or different heteroatoms, such as nitrogen, oxygen and sulfur, and which is unsubstituted or monosubstituted or polysubstituted by Hal, A, Ar′, OR⁵, COOR⁵, CN, N(R⁵)₂, NO₂, NHCOA, NHCOAr′ and/or carbonyl oxygen,

Hal is F, Cl, Br or I,

m is 0, 1, 2, 3 or 4,

n is 0, 1 or 2,

and salts thereof,

which are described in WO 9931092,

c) the compounds of the formula I

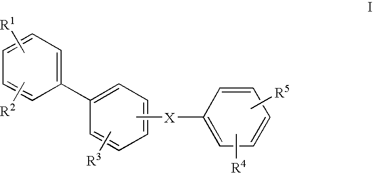

in which

R¹ and R⁴ are each, independently of one another, —C(═NH)—NH₂, which may also be monosubstituted by —COA, —CO-[C(R⁶)₂]ₙ—Ar, —COOA, —OH or by a conventional amino-protecting group,

or are NH—C(═NH)—NH₂, —CO—N═C(NH₂)₂,

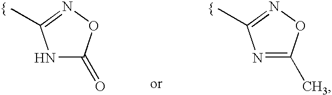

R², R³

and R⁵ are each, independently of one another, H, A, OR⁶, N(R⁶)₂, NO₂, CN, Hal, NHCOA, NHCOAr, NHSO₂A, NHSO₂Ar, COOR⁶, CON(R⁶), CONHAr, COR⁶, COAr, S(O)ₙA, S(O)Ar, —O-[C(R⁶)₂]ₘ—COOR⁶, —[C(R⁶)₂]ₚ—COOR⁶, —O—[C(R⁶)]ₘ—CON(R⁶)₂, —[C(R⁶)₂]ₚ—CON(R⁶)₂, —O—[C(R⁶)₂]ₘ—CONHAr or —[C(R⁶)₂]ₚ—CONHAr,

X is —[C(R⁶)₂]ₙ—, —CR⁶═CR⁶—, —[C(R⁶)₂]ₙ—O—, —O—[C(R⁶)₂]ₙ—, —COO—, —OOC—, —CONR⁶— or —NR⁶CO—,

R⁶ is H, A or benzyl,

A is alkyl having 1-20 carbon atoms, in which one or two CH₂ groups may be replaced by O or S atoms or by —CR⁶═CR⁶— groups and/or 1-7H atoms may be replaced by F,

Ar is phenyl or naphthyl, each of which is unsubstituted or monosubstituted, disubstituted or trisubstituted by A, Ar′, OR⁶, OAr′, N(R⁶)₂, NO₂, CN, Hal, NHCOA, NHCOAr′, NHSO₂A, NHSO₂Ar′, COOR⁶, CON(R⁶)₂, CONHAr′, COR⁶, COAr′, S(O)ₙA or S(O)ₙAr′,

Ar′ is phenyl or naphthyl, each of which is unsubstituted or monosubstituted, disubstituted or trisubstituted by A, OR⁶, N(R⁶)₂, NO₂, CN, Hal, NHCOA, COOR⁶, CON(R⁶)₂, COR⁶ or S(O)ₙA,

Hal is F, Cl, Br or I,

n is 0, 1 or 2,

m is 1 or 2,

p is 1 or 2,

and salts thereof,

which are described in WO 9957096,

d) the compounds of the formula I

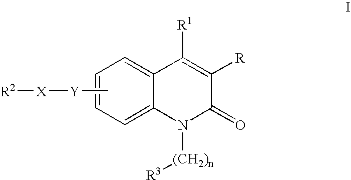

in which

R and R¹ are each, independently of one another, H, A, —(CH₂)ₘ—R⁴, —(CH₂)ₘ—OA or —(CH₂)ₘ—Ar,

R²

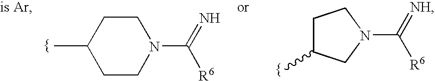

R³ is Ar,

R⁴ is CN, COOH, COOA, CONH₂, CONHA, CONA₂ or C(═NH)—NH₂,

R⁵ is —C(═NH)—NH₂, —NH—C(═NH)—NH₂ or —C(═O)—N═C(NH₂)₂, each of which is unsubstituted or monosubstituted by —COA, —COOA, —OH or by a conventional amino-protecting group, or is

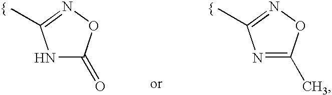

R⁶ is H, A or NH₂,

Ar is phenyl, naphthyl or biphenyl, each of which is unsubstituted or monosubstituted, disubstituted or trisubstituted by A, cycloalkyl having 3-6 carbon atoms, OH, OA, Hal, CN, NO₂, CF₃, NH₂, NHA, NA₂, pyrrolidin-1-yl, piperidin-1-yl, benzyloxy, SO₂NH₂, SO₂NHA, SO₂NA₂, —(CH₂)ₙ—NH₂, —(CH₂)ₙ—NHA, —(CH₂)ₙ—NA₂, —O—(CH₂)ₙ—NH₂, —O—(CH₂)ₙ—NHA, —O—(CH₂)ₙ—NA₂, —O—(CH₂)ₘ—O— or R⁵,

A is alkyl having. 1-6 carbon atoms,

X is absent or is alkylene having 14 carbon atoms or carbonyl,

Y is absent or is NH, O or S,

Hal is F, Cl, Br or I,

m is 0, 1 or 2,

n is 0, 1, 2 or 3,

and salts thereof,

which are described in WO 0012479,

e) the compounds of the formula I

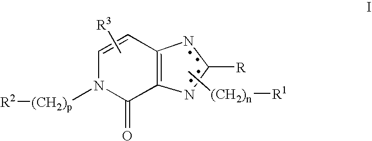

in which

R is H, unbranched or branched alkyl having 1-6 carbon atoms or cycloalkyl having 3-6 carbon atoms,

R¹ is Ar,

R² is Ar′,

R³ is H, R, R⁴, Hal, CN, COOH, COOA or CONH₂,

Ar and Ar′ are each, independently of one another, phenyl, naphthyl or biphenyl, each of which is unsubstituted or monosubstituted, disubstituted or trisubstituted by R, OH, Hal, CN, NO₂, CF₃, NH₂, NHR, NR₂, pyrrolidin-1-yl, piperidin-1-yl, benzyloxy, SO₂NH₂, SO₂NHR, SO₂NR₂, —CONHR, —CONR₂, —(CH₂)ₙ—NH₂, —(CH₂)ₙ—NHR, —(CH₂)ₙ—NR₂, —O—(CH₂)ₙ—NH₂, —O—(CH₂)ₙ—NHR, —O—(CH₂)ₙ—NR₂, R⁴ or together by —O—(CH₂)ₘ—O—,

R⁴ is —C(═NH)—NH₂, —NH—C(═NH)—NH₂ or —C(═O)—N═C(NH₂)₂, each of which is unsubstituted or monosubstituted by —COR, —COOR, —OH or by a conventional amino-protecting group, or is

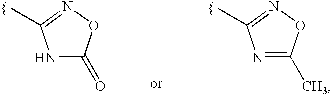

A is alkyl having 1-4 carbon atoms,

Hal is F, Cl, Br or I,

m is 1 or 2,

n is 0, 1, 2 or 3,

p is 0 or 1,

and salts thereof,

which are described in WO 0020416,

f) the compounds of the formula I

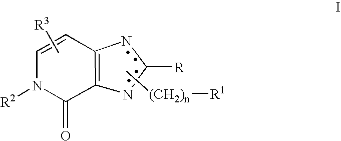

in which

R is H, unbranched or branched alkyl having 1-6 carbon atoms or cycloalkyl having 3-6 carbon atoms,

R¹ is Ar,

R² is Ar′,

R³ is H, R, R⁴, Hal, CN, COOH, COOA or CONH₂,

Ar and Ar′ are each, independently of one another, phenyl, naphthyl or biphenyl, each of which is unsubstituted or monosubstituted, disubstituted or trisubstituted by R, OH, Hal, CN, NO₂, CF₃, NH₂, NHR, NR₂, pyrrolidin-1-yl, piperidin-1-yl, benzyloxy, SO₂NH₂, SO₂NHR, SO₂NR₂, —CONHR, —CONR₂, —(CH₂)ₙ—NH₂, —(CH₂)ₙ—NHR, —(CH₂)ₙ—NR₂, —O—(CH₂)ₙ—NH₂, —O—(CH₂)ₙ—NHR, —O—(CH₂)ₙ—NR₂, R⁴ or together by —O—(CH₂)ₘ—O—, or isoquinolinyl which is substituted by NH₂,

R⁴ is —C(═NH)—NH₂, —NH—C(═NH)—NH₂ or —C(═O)—N═C(NH₂)₂, each of which is unsubstituted or monosubstituted by —COR, —COOR, —OH or by a conventional amino-protecting group, or is

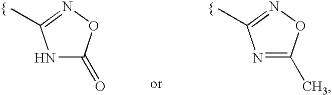

A is alkyl having 14 carbon atoms,

Hal is F, Cl, Br or I,

m is 1 or 2,

n is 0 or 1,

and salts and solvates thereof,

which are described in WO 0040583,

g) the compounds of the formula I

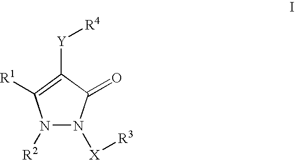

in which

R¹ and R² are each, independently of one another, H, A, cycloalkyl-[C(R⁷R⁷⁴⁰)]ₙ— or Ar—[C(R⁷R^{7′})]ₙ—,

R³ and R⁴ are each, independently of one another, H, Ar, Het or R⁵, where at least one of the two radicals is R⁵,

R⁵ is phenyl, naphthyl or biphenyl, each of which is substituted by —C(═NH₁—NH₂, which may also be monosubstituted by —COA, Ar-[C(R⁷R⁷)]ₙ—CO—, COOA, OH or by a conventional amino-protecting group, —NH—C(═NH)—NH₂, —CO—N═C(NH₂)₂,

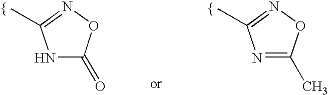

and which may optionally additionally be monosubstituted or disubstituted by A, Ar′, Het, OR⁶, NR⁶R^{6′}, NO₂, CN, Hal, NR⁶COA, NR⁶COAr′, NR⁶SO₂A, NR⁶SO₂Ar′, COOR⁶, CO—NR⁶R⁶, COR⁷, CO—Ar′, SO₂NR⁶R^{6′}, S(O)ₙAr′ or S(O)ₙA,

R⁶ and R^{6′} are each, independently of one another, H, A, CR⁷R^{7′}—Ar or CR⁷R^{7′}-Het,

R⁷ and R^{7′} are each, independently of one another, H or A,

X and Y are each, independently of one another, (CR⁷R^{7′})ₙ,

A is alkyl having 1-20 carbon atoms, in which one or two CH₂ groups may be replaced by O or S atoms and/or by —CH═CH— groups and/or in addition 1-7H atoms may be replaced by F,

Ar is phenyl, naphthyl or biphenyl, each of which is unsubstituted or monosubstituted, disubstituted or trisubstituted by A, Ar, Het, OR⁶₉ NR⁶R^{6′}, NO₂, CN, Hal, NR⁶COA, NR⁶COAr′, NR⁶SO₂A, NR⁶SO₂Ar′, COOR⁶, CO—NR⁶R^{6′}, CON⁶Ar′, COR⁷, COAr′, SO₂NR⁶R^{6′}, S(O)Ar′ or S(O)ₙA,

Ar′ is phenyl or naphthyl, each of which is unsubstituted or monosubstituted, disubstituted or trisubstituted by A, OR⁷, NR⁷R^{7′}, NO₂, CN, Hal, NR⁷COA, NR⁷SO₂A, COOR⁷, CO—NR⁷R^{7′}, COR⁷, SO₂NR⁷R^{7′} or S(O)ₙA,

Het is a monocyclic or bicyclic, saturated, unsaturated or aromatic heterocyclic radical having from 1 to 4 N, O and/or S atoms, which may be unsubstituted or monosubstituted, disubstituted or trisubstituted by A, OR⁷, NR⁷R^{7′}, NO₂, CN, Hal, NR⁷COA, NR⁷SO₂A, COOR⁷, CO—NR⁷R^{7′}, COR⁷, SO₂NR⁷R^{7′}, S(O)ₙA and/or carbonyl oxygen,

Hal is F, Cl, Br or I,

n is 0, 1 or 2,

and their pharmaceutically tolerated salts and solvates,

which are described in WO 0051989,

h) compounds of the formula I

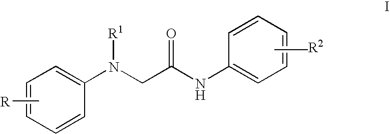

in which

R is —CO—N═C(NH₂)₂, —NH—C(═NH)—NH₂ or —C(═NH)—NH₂, which may also be monosubstituted by OH, —OCOOA, —OCOO(CH₂)ₙNAA′, —COO(CH₂)ₙNAA′, —OCOO(CH₂)ₘ-Het, —COO(CH₂)ₘ-Het, —CO—CAA′—R³, —COO—CAA′—R³, COOA, COSA, COOAr, COOAr or by a conventional amino-protecting group, or is

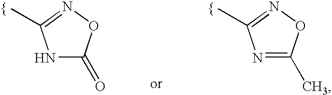

R¹ is unbranched, branched or cyclic alkyl having 1-20 carbon atoms, in which one or two CH₂ groups may be replaced by O or S atoms, or is Ar, Ar or X,

R² is phenyl which is monosubstituted by S(O)ₚA, S(O)ₚNHA, CF₃, COOA, CH₂NHA, CN or OA,

R³

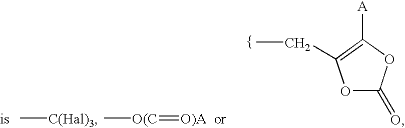

Ar is phenyl or naphthyl, each of which is unsubstituted or monosubstituted, disubstituted or trisubstituted by A, OA, NAA′, NO₂, CF₃, CN, Hal, NHCOA, COOA, CONAA′, S(O)ₚA or S(O)ₚNAA′,

Ar is —(CH₂)ₙ—Ar,

A and A′ are each, independently of one another, H or unbranched, branched or cyclic alkyl having 1-20 carbon atoms,

Het is a monocyclic or bicyclic, saturated, unsaturated or aromatic heterocyclic radical having from 1 to 4 N, O and/or S atoms, bonded via N or C, which may be unsubstituted or substituted by A,

X is —(CH₂)ₙ—Y,

Y

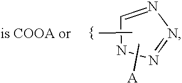

Hal is F, Cl, Br or I,

m is 0 or 1,

n is 1, 2, 3, 4, 5 or 6,

p is 0, 1 or 2,

and their pharmaceutically tolerated salts and solvates,

i) compounds of the formula I

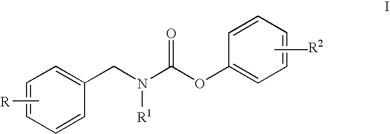

in which

R is —CO—N═C(NH₂)₂, —NH—C(═NH)—NH₂ or —C(═NH)—NH₂, which may also be monosubstituted by OH, —OCOOA, —OCOO(CH₂)ₙNAA′, —COO(CH₂)ₙNAA′, —OCOO(CH₂)ₘ-Het, —COO(CH₂)ₘ-Het, —CO—CAA′—R³, —COO—CAA′—R³, COOA, COSA, COOAr, COOAr′ or by a conventional amino-protecting group, or is

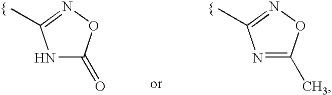

R¹ is unbranched, branched or cyclic alkyl having 1-20 carbon atoms, in which one or two CH₂ groups may be replaced by O or S atoms, or is Ar, Ar′ or X,

R² is phenyl which is monosubstituted by S(O)ₚA, S(O)ₚNHA, CF₃, COOA, CH₂NHA, CN or OA,

R³ is —C(Hal)₃, —O(C═O)A or

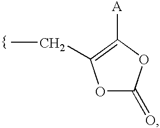

Ar is phenyl or naphthyl, each of which is unsubstituted or monosubstituted, disubstituted or trisubstituted by A, OA, NAA′, NO₂, CF₃, CN, Hal, NHCOA, COOA, CONAA′, S(O)ₚA or S(O)ₚNAA′,

Ar′ is —(CH₂)ₙ—Ar,

A and A′ are each, independently of one another, H or unbranched, branched or cyclic alkyl having 1-20 carbon atoms,

Het is a monocyclic or bicyclic, saturated, unsaturated or aromatic heterocyclic radical having from 1 to 4 N, O and/or S atoms, bonded via N or C, which may be unsubstituted or substituted by A,

X is —(CH₂)ₙ—Y,

Y

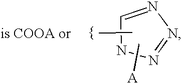

Hal is F, Cl, Br or I,

m is 0 or 1,

n is 1, 2, 3, 4, 5 or 6,

p is 0, 1 or 2,

and their pharmaceutically tolerated salts and solvates,

j) compounds of the formula I

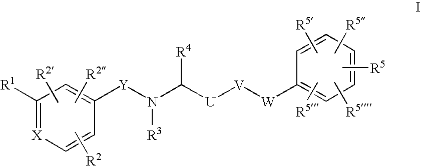

in which

R¹ is H, Cl, F, OH, OA, O—(CH₂)ₙ—Ar, NH₂, NHCOA, NHCOOA, NH—(CH₂)ₙ—Ar, CN, CONH₂, CSNH₂, C(═NH)SA, C(═NH)NH₂, C(═NH—OH)—NH₂, C(═NH—O—COA)-NH₂, C(═NH—O—COAr)NH₂, C(═NH—O—COHet)-NH₂, C(═NH)—OA, C(═NH)NHNH₂, C(═NH)NHNHA, C(═NH)NH—COOA, C(═NH)NH—COA, C(═NH)NH—COO—(CH₂)ₘ—Ar, C(═NH)NH—COO—(CH₂)ₘ—Het, NH—C(═NH)NH₂, NH—C(═NH )NH—COOA, NHC(═NH)NH—COO—(CH₂)ₘ—Ar,

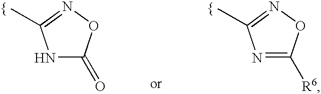

R² R^{2′}

and R² are each, independently of one another, H, A, CF₃, Cl, F, COA, COOH, COOA, CONH₂, CONHA, CONA₂, CH₂NH₂, CH₂NHCOA, CH₂NHCOOA, OH, OA, OCF₃, NO₂, SO₂A, SO₂NH₂ or SO₂NHA,

R³ and R⁴ together are (CH₂)ₚ, CO(CH₂)ₚ, COO(CH₂)ₙ, COOCH(A)-, COOCH(Ar)—, CONH(CH₂)ₙ, CH₂CH(OR⁷)—(CH₂)ₙ—, CH₂—O—(CH₂)ₙ, CH₂—S—(CH₂)ₙ, CA₂—O—(CH₂)ₙ, CA₂—S—(CH₂)ₙ, CHAr—S—(CH₂)ₙ, (CH₂)₂NHCH₂ or (CH₂)₂—N(R⁸)—CH₂,

R⁵, R^{5′}, R^{5″},

R^{5′″} and R^{5″″} are each, independently of one another, (CH₂)ₙ—COOH, (CH₂)ₙ—COO—(CH₂)ₙ—Ar, Ar, Py or R²,

R⁶ is OH, A or Ar,

R⁷ is H, A, Ar or Het,

R⁸ is H, (CH₂)ₙ—COOH, (CH₂)ₘ—COOA, (CH₂)ₘ—COO—(CH₂)ₙ—Ar, (CH₂)ₘ—COO—(CH₂)ₙ-Het, (CH₂)ₘ—CONH₂, (CH₂)ₘ—CONHA, (CH₂)ₘ—CONA₂, A, COA, SO₂A or SO₃H,

R⁹ is H, A or benzyl,

U is CO or CH₂,

V is NH or CO,

W is absent or is CO,

X is CH or N,

Y is absent or is CH₂, CO or SO₂,

A is unbranched, branched or cyclic alkyl having 1-20 carbon atoms, in which one or two CH₂ groups may be replaced by O or S atoms, —CH═CH— or —C≡C— and/or 1-7H atoms may be replaced by F,

Ar is phenyl or naphthyl, each of which is unsubstituted or monosubstituted, disubstituted or trisubstituted by A, CF₃, Hal, OH, OA, OCF₃, SO₂A, SO₂NH₂, SO₂NHA, SO₂NA₂, NH₂, NHA, NA₂, NHCHO, NHCOA, NHCOOA, NACOOA, NHSO₂A, NHSO₂Ar, COOH, COOA, COO—(CH₂)ₘ—Ar′, COO—(CH₂)ₘ-Het, CONH₂, CONHA, CONA₂, CONHAr′CHO, COA, COAr′, CH₂Ar′, (CH₂)ₘNH₂, (CH₂)ₘNHA, (CH₂)ₘNA₂, (CH₂)ₘNHCHO, (CH₂)ₘNHCOA, (CH₂)ₘNHCOOA, (CH₂)ₘNHCOO—(CH₂)ₘAr′, (CH₂)ₘNHCOO—(CH₂)ₘHet, NO₂, CN, CSNH₂, C(═NH)SA, C(═NH)OA, C(═NH)NH₂, C(═NH)NHOH, C(═NH)NHCOOA or C(═NH)NHCOOAr′,

Ar′ is phenyl or naphthyl, each of which is unsubstituted or monosubstituted, disubstituted or trisubstituted by A, OR⁹, N(R⁹)₂, NO₂, CN, Hal, NHCOA, COOR⁹, CON(R⁹)₂, COR⁹ or S(O)₂A,

Het is a monocyclic or bicyclic, saturated, unsaturated or aromatic heterocyclic radical having 1-4 N, O and/or S atoms, bonded via N or C, which is unsubstituted or monosubstituted, disubstituted, trisubstituted or tetrasubstituted by A, CF₃, Hal, OH, OA, OCF₃, SO₂A, SO₂—(CH₂)ₘ—Ar, SO₂NH₂, SO₂NHA, SO₂NA₂, NH₂, NHA, NA₂, NHCHO, NHCOA, NHCOOA, NACOOA, NHSO₂A, NHSO₂Ar, COOH, COOA, COO—(CH₂)ₘ—Ar′, CONH₂, CONHA, COA, COAr′, CH₂NH₂, CH₂NHA, CH₂NHCHO, CH₂NHCOA, CH₂NHCOOA, NO₂, CN, CSNH₂, C(═NH)SA, C(═NH)OA, C(═NH)NH₂, C(═NH)NHOH, C(═NH)NHCOOA, C(═NH)COOAr′ and/or carbonyl oxygen,

Py is 2-, 3- or 4-pyridyl, each of which is unsubstituted or monosubstituted or polysubstituted by A, Hal, CN, CONH₂, CONHA, COOH, COOA, CH₂NH₂, CH₂NHA, CH₂NHCHO, CH₂NHCOA, CH₂NHCOOA, CH₂OH, CH₂OA, CH₂OAr, CH₂OCOA, NO₂, NH₂, NHA or NA₂,

Hal is F, Cl, Br or I,

n is 1 or 2,

m is 0, 1 or 2,

p is 2, 3 or 4,

and their pharmaceutically tolerated salts and solvates,

k) compounds of the formula I

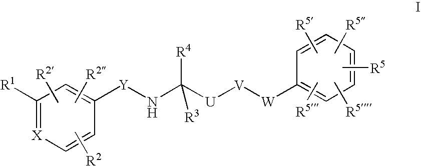

in which

R¹ is H, Cl, F, OH, OA, O—(CH₂)ₙ—Ar, NH₂, NHCOA, NHCOOA, NH—(CH₂)ₙ—Ar, CN, CONH₂, CSNH₂, C(═NH)SA, C(═NH)NH₂, C(═NH—OH)—NH₂, C(═NH—O—COA)-NH₂, C(═NH—O—COAr)—NH₂, C(═NH—O—COHet)-NH₂, C(═NH)—OA, C(═NH)NHNH₂, C(═NH)NHNHA, C(═NH)NH—COOA, C(═NH)NH—COA, C(═NH)NH—COO—(CH₂)ₘ—Ar, C(═NH)NH—COO—(CH₂)ₘ-Het, NH—C(═NH)NH₂, NH—C(═NH)NH—COOA, NHC(═NH)NH—COO—(CH₂)ₘ—Ar,

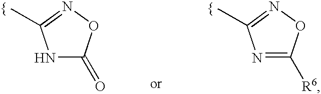

R², R^{2′}

and R^{2″} are each, independently of one another, H, A, CF₃, Cl, F, COA, COOH, COOA, CONH₂, CONHA, CONA₂, CH₂NH₂, CH₂NHCOA, CH₂NHCOOA, OH, OA, OCF₃, NO₂, SO₂A, SO₂NH₂, SO₂NHA or SO₂NA₂,

R³ is A, (CH₂)ₙ—Ar or (CH₂)ₙ-Het,

R⁴ is A,

R³ and R⁴ together are alternatively (CH₂)ₚ, (CH₂)ₙ—N(R⁸)—(CH₂)₂, (CH₂)₂—CH(NH₂)—(CH₂)₂—, (CH₂)₂—CH(NH—COOA)-(CH₂)₂—, (CH₂)₂—CH(NH—CH₂—COOA)-(CH₂)₂—, (CH₂)₂—CH[NH—CH(A)-COOA]-(CH₂)₂—, (CH₂)₂—O—(CH₂)₂, (CH₂)₂—S(O)ₘ—(CH₂)₂ or

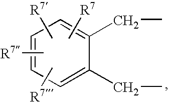

R⁵, R^{5′}, R^{5″}

R^{5′″} and R^{5″″} are each, independently of one another, (CH₂)ₙ—COOH, (CH₂)ₙ—COOA, (CH₂)ₙ—COO—(CH₂)ₘ—Ar, (CH₂)ₙ—COO—(CH₂)ₘ-Het, Ar, Py or R²,

R⁶ is OH, A or Ar,

R⁷, R^{7′}, R^{7″}

and R^{7′″} are each, independently of one another, H, Hal, OH, OA, COOH, COOA, COO(CH₂)ₘAr, CONH₂, CONHA or CONA₂,

R⁸ is H, A, COA, COOA, (CH₂)ₙ—COOH, (CH₂)ₘ—COOA, COO—(CH₂)ₘ—Ar, COO—(CH₂)ₘ-Het, (CH₂)ₙ—COO—(CH₂)ₘ—Ar, (CH₂)ₙ—COO—(CH₂)ₘ-Het, (CH₂)ₘ—CONH₂, (CH₂)ₘ—CONHA, (CH₂)ₘ—CONA₂, SO₂A or SO₃H,

R⁹ is H, A or benzyl,

U is CO or CH₂,

V is NH or CO,

W is absent or is CO,

X is CH or N,

Y is absent or is CH₂, CO or SO₂,

A is unbranched, branched or cyclic alkyl having 1-20 carbon atoms, in which one or two CH₂ groups may be replaced by O or S atoms, —CH═CH— or —C≡C— and/or 1-7H atoms may be replaced by F,

Ar is phenyl or naphthyl, each of which is unsubstituted or monosubstituted, disubstituted or trisubstituted by A, CF₃, Hal, OH, OA, OCF₃, SO₂A, SO₂NH₂, SO₂NHA, SO₂NA₂, NH₂, NHA, NA₂, NHCHO, NHCOA, NHCOOA, NACOOA, NHSO₂A, NHSO₂Ar, COOH, COOA, COO—(CH₂)ₘ—Ar′, COO—(CH₂)ₘ-Het, CONH₂, CONHA, CONA₂, CONHAr′, CHO, COA, COAr′, CH₂Ar′, (CH₂)ₘNH₂, (CH₂)ₘNHA, (CH₂)ₘNA₂, (CH₂)ₘNHCHO, (CH₂)ₘNHCOA, (CH₂)ₘNHCOOA, (CH₂)ₘNHCOO—(CH₂)ₘAr′, (CH₂)ₘNHCOO—(CH₂)ₘHet, NO₂, CN, CSNH₂, C(═NH)SA, C(═NH)OA, C(═NH)NH₂, C(═NH)NHOH, C(═NH)NHCOOA or C(═NH)NHCOOAr′,

Ar′ is phenyl or naphthyl, each of which is unsubstituted or monosubstituted, disubstituted or trisubstituted by A, OR⁹, N(R⁹)₂, NO₂, CN, Hal, NHCOA, COOR⁹, CON(R⁹)₂, COR⁹ or S(O)₂A,

Het is a monocyclic or bicyclic, saturated, unsaturated or aromatic heterocyclic radical having 1-4 N, O and/or S atoms, bonded via N or C, which is unsubstituted or monosubstituted, disubstituted, trisubstituted or tetrasubstituted by A, CF₃, Hal, OH, OA, OCF₃, SO₂A, SO₂—(CH₂)ₘ—Ar, SO₂NH₂, SO₂NHA, SO₂NA₂, NH₂, NHA, NA₂, NHCHO, NHCOA, NHCOOA, NACOOA, NHSO₂A, NHSO₂Ar, COOH, COOA, COO—(CH₂)ₘ—Ar′, CONH₂, CONHA, COA, COAr′, CH₂NH₂, CH₂NHA, CH₂NHCHO, CH₂NHCOA, CH₂NHCOOA, NO₂, CN, CSNH₂, C(═NH)SA, C(═NH)OA, C(═NH)NH₂, C(═NH)NHOH, C(═NH)NHCOOA, C(═NH)COOAr′ and/or carbonyl oxygen,

Py is 2-, 3- or 4-pyridyl, each of which is unsubstituted or monosubstituted or polysubstituted by A, Hal, CN, CONH₂, CONHA, COOH, COOA, CH₂NH₂, CH₂NHA, CH₂NHCHO, CH₂NHCOA, CH₂NHCOOA, CH₂OH, CH₂OA, CH₂OAr, CH₂OCOA, NO₂, NH₂, NHA or NA₂,

Hal is F, Cl, Br or I,

n is 1 or 2,

m is 0, 1 or 2,

p is 2, 3, 4-or 5,

and their pharmaceutically tolerated salts and solvates,

l) compounds of the formula I

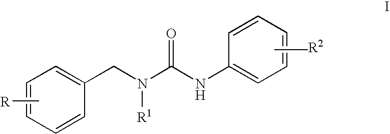

in which

R is CN, CH₂NH₂, —NH—C(═NH)—NH₂, —CO—N═C(NH₂)₂, —C(═NH)—NH₂, which may also be monosubstituted by Ar′, OH, O—COA, O—COAr, OCOOA, OCOO(CH₂)ₙN(A)₂, —COO(CH₂)ₙNA₂, OCOO(CH₂)ₘHet, COO—(CH₂)ₘ-Het, CO—C(A)₂—R³, COOA, COSA, COSAr, COOAr, COOAr′, COA, COAr, COAr′ or by a conventional amino-protecting group, or is

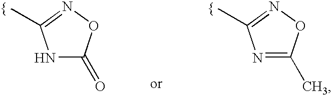

R¹ is R⁴, Ar, Ar′ or X,

R² is phenyl which monosubstituted by SA, SOA, SO₂A, SONHA, SO₂NHA, CF₃, COOA, CH₂NHA, CN or OA,

R³

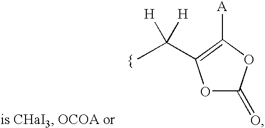

R⁴ is alkyl having 1-20 carbon atoms, in which one or two CH₂ groups may be replaced by O or S atoms and/or by —CH═CH— groups and/or in addition 1-7H atoms may be replaced by F,

A is H or alkyl having 1-20 carbon atoms,

A′ is alkyl having 1-10 carbon atoms,

Ar is phenyl or naphthyl, each of which is unsubstituted or monosubstituted, disubstituted or trisubstituted by A′, OH, OA′, NH₂, NHA′, NA′₂, NO₂, CF₃, CN, Hal, NHCOA, COOA, CONH₂, CONHA′, CONA′₂, SA, SOA, SO₂A,

SO₂NH₂, SO₂NHA′ or SO₂NA′₂,

Ar′ is (CH₂)ₙ—Ar,

Het is a monocyclic or bicyclic, saturated, unsaturated or aromatic heterocyclic radical having from 1 to 4 N, O and/or S atoms, which may be unsubstituted or monosubstituted, disubstituted or trisubstituted by A′, OA′, NH₂, NHA′, NA′₂, NO₂, CN, Hal, NHCOA′, NHSO₂A′, COOA, CONH₂, CONHA′, CONA′₂, COA, SO₂NH₂, SA′, SOA′, SO₂A′ and/or carbonyl oxygen,

X is (CH₂)ₙY,

Y

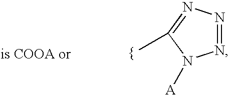

Hal is F, Cl, Br or I,

n is 1, 2, 3, 4, 5 or 6,

m is 0 or 1,

and their pharmaceutically tolerated salts and solvates,

m) compounds of the formula I

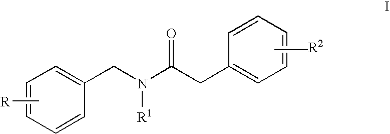

in which

R is CH₂NH₂, —CO—N═C(NH₂)₂, —NH—C(═NH)—NH₂ or —C(═NH)—NH₂, which may also be monosubstituted by OH, —OCOOA, —OCOO(CH₂)ₙNAA′, —COO(CH₂)ₙNAA′, —OCOO(CH₂)ₘ-Het, —COO(CH₂)ₘ-Het, —CO—CAA′—R³, —COO—CAA′—R³, COOA, COSA, COOAr, COOAr′ or by a conventional amino-protecting group, or is

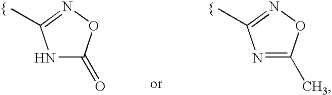

R¹ is unbranched, branched or cyclic alkyl having 1-20 carbon atoms, in which one or two CH₂ groups may be replaced by O or S atoms, or is Ar, Ar′ or X,

R² is phenyl which is monosubstituted by S(O)ₚA, S(O)ₚNHA, CF₃, COOA, CH₂NHA, CN or OA,

R³

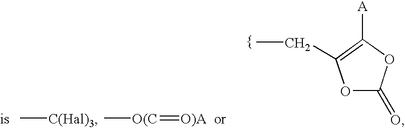

Ar is phenyl or naphthyl, each of which is unsubstituted or monosubstituted, disubstituted or trisubstituted by A, OA, NAA′, NO₂, CF₃, CN, Hal, NHCOA, COOA, CONAA′, S(O)ₚA or S(O)ₚNAA′,

Ar′ is —(CH₂)ₙ—Ar,

A is H or unbranched, branched or cyclic alkyl having 1-20 carbon atoms,

A′ is unbranched, branched or cyclic alkyl having 1-10 carbon atoms,

Het is a monocyclic or bicyclic, saturated, unsaturated or aromatic heterocyclic radical having from 1 to 4 N, O and/or S atoms, bonded via N or C, which may be unsubstituted or substituted by A,

X is —(CH₂)ₙ—Y,

Y

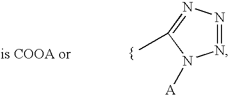

Hal is F, Cl, Br or I,

m is 0 or 1,

n is 1, 2, 3, 4, 5 or 6,

p is 0, 1 or 2,

and their pharmaceutically tolerated salts and solvates,

n) compounds of the formula I

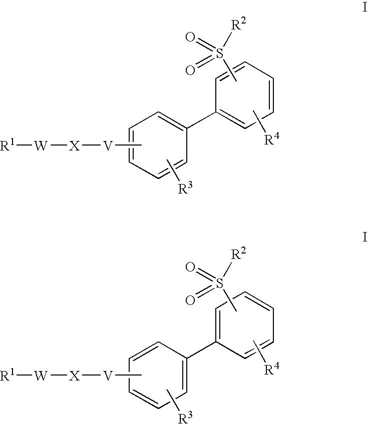

in which:

R¹ is phenyl or naphthyl, each of which is substituted by —C(═NH)NH₂, which may also be monosubstituted by —COA, —CO—[C(R⁶)₂—Ar′, —COOA, —OH or by a conventional amino-protecting group, —NHC(═NH)—NH₂,

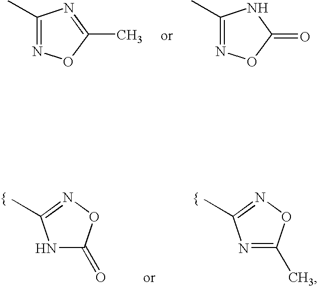

and which may optionally be substituted by -A, —OR⁵, —N(R⁵)₂, —NO₂, —CN, -Hal, —NR⁵COA, —NR⁵COAr′, —NR⁵SO₂A, —NR⁵SO₂Ar′, —COOR⁵, —CON(R⁵)₂, —CONR⁵Ar′, —COR⁶, —COAr′ or S(O)ₙA;

R² is —N(R⁵)₂, —NR⁵COA, —NR⁵COAr or —NR⁵COOR⁵,

R³ and

R⁴, independently of one another, are —H, -A, —OR⁵, —N(R⁵)₂, —NO₂, —CN, -Hal, —NR⁵COA, —NR⁵COAr, —NR⁵SO₂A, —NR⁵SO₂Ar′, —COOR⁵, —CON(R⁵)₂, —CONR⁵Ar′, —COR⁶, —COAr′, —S(O)Ar′ or S(O)ₙA;

R⁵— is H, -A, —C(R⁶R⁷)Ar′ or —C(R⁶R⁷)Het;

R⁶ and

R⁷, independently of one another, are —H, -A or —(CH₂)₁—Ar′;

R⁸ is H or A;

X is —O—, —NR⁵—, —CONR⁵—, —N(SO₂Ar)— or —N(SO₂Het)-;

W is —(CR⁶R⁷)ₙ—, —OCR⁶R⁷—, 1,3-phenylene, 1,3-phenylene- —C(R⁶)₂—, 1,4-phenylene or 1,4-phenylene-C(R⁶)₂—;

V is —(C(R⁶)₂)ₘ—;

A is alkyl having from 1 to 20 carbon atoms, in which one or two CH₂ groups may be replaced by O or S atoms or by —CH═CH— groups and in addition by from 1 to 7H atoms may be replaced by F;

Ar′ is phenyl or naphthyl, each of which is unsubstituted or monosubstituted, disubstituted or trisubstituted by -A, —Ar′, -Het, —OR⁵, —N(R⁵)₂, —NO₂, —CN, -Hal, —NR⁵COA, —NR⁵COAr, —NR⁵SO₂A, —NR⁵SO₂Ar′, —COOR⁵, —CON(R⁵)₂, —CONR⁵Ar′, —COR⁶, —COAr′ or —S(O)ₙA,

Ar′ is phenyl or naphthyl, each of which is unsubstituted or monosubstituted, disubstituted or trisubstituted by -A, —OR⁶, —N(R⁶)₂, —NO₂, —CN, -Hal, —NR⁶COA, —NR⁶SO₂A, —COOR, —CON(R⁶)₂—COR⁶, —SO₂NR⁶ or —S(O)ₙA,

Het is a monocyclic or bicyclic, saturated, unsaturated or aromatic heterocyclic radical having from 1 to 4 N, O and/or S atoms, bonded via N or C, which may be unsubstituted or monosubstituted, disubstituted or trisubstituted by -A, —OR⁶, —N(R⁶)₂, —NO₂, —CN, -Hal, —NR⁶COA, —NR⁶SO₂A, —COOR⁶, —CON(R⁶)₂, —COR⁶, —SO₂NR⁶, —S(O)ₙA and/or carbonyl oxygen;

Hal is —F, —Cl, —Br or —I;

I is 0, 1, 2, 3, 4 or 5;

m is 0 or 1;

n is 0, 1 or 2;

and their pharmaceutically tolerated salts and solvates,

o) compounds of the formula I

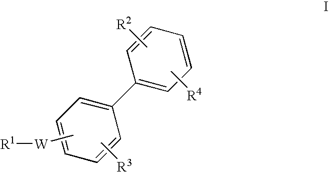

in which

R¹ is phenyl or naphthyl, each of which is substituted by —C(═NH)NH₂, which may also be monosubstituted by —COA, —CO-[C(R⁷)₂]ₙ—Ar′, —COOA, —OH or by a conventional amino-protecting group, —NHC(═NH)—NH₂, —CON═C(NH₂)₂,

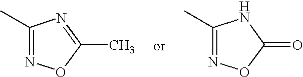

and which may optionally be substituted by -A, —OR⁵, —N(R⁵)₂, —NO₂, —CN, -Hal, —NR⁵COA, —NR⁵COAr′, —NR⁵SO₂A, —NR⁵SO₂Ar′, —COOR⁵, —CON(R⁵)₂, —COR⁷, —COAr′ or S(O)ₙA;

R² is —S(O)ₙA, —CF₃, —COOR or —OA;

R³ and

R⁴, independently of one another, are —H, -A, —OR⁵, —N(R⁵)₂A, —NO₂, —CN, -Hal, —NR⁵COA, —NR⁵COAr′, —NR⁵SO₂A, —NR⁵SO₂Ar′, —COOR⁵, —CON(R⁵)₂, —CONR⁵Ar, —COR⁷, —COAr′ or —S(O)ₙA;

R⁵ and

R⁶, independently of one another, are —H, -A, —[C(R⁷R⁸)]ₙAr′ or —[C(R⁷R⁸)]ₙHet;

R⁷ and

R⁸, independently of one another, are —H or -A;

W is —[C(R⁵R⁶)]ₘCONR⁵[C(R⁵R⁶)]₁— or —OC(R⁵R⁶)CONR⁵[C(R⁵R⁶)]₁—;

A is alkyl having from 1 to 20 carbon atoms, in which one or two CH₂ groups may be replaced by O or S atoms or by —CH═CH— groups and in addition from 1 to 7H atoms may be replaced by —F;

Ar is phenyl or naphthyl, each of which is unsubstituted or monosubstituted, disubstituted or trisubstituted by -A, —Ar′, -Het, —OR⁵, —N(R⁵)₂, —NO₂, —CN, -Hal, —NR⁵COA, —NR⁵COAr, —NR⁵SO₂A, —NR⁵SO₂Ar′, —COOR⁵, —CON(R⁵)₂, —CONR⁵Ar′, —COR⁷, —COAr′, —SO₂NR⁵, —S(O)ₙAr′ or —S(O)ₙA;

Ar′ is phenyl or naphthyl, each of which is unsubstituted or monosubstituted, disubstituted or trisubstituted by -A, —OR⁷, —N(R⁷)₂, —NO₂, —CN, -Hal, —NR⁷COA, —NR⁷SO₂A, —COOR⁷, —CON(R⁷)₂, —COR⁷, —SO₂NR⁷ or —S(O)ₙA;

Het is a monocyclic or bicyclic, saturated, unsaturated or aromatic heterocyclic radical having from 1 to 4 N, O and/or S atoms, bonded via N or C, which may be unsubstituted or monosubstituted, disubstituted or trisubstituted by -A, —OR⁷, —N(R⁷)₂, —NO₂, —CN, -Hal, —NR⁷COA, —NR⁷SO₂A, —COOR⁷, —CON(R⁷)₂, —COR⁷, —SO₂NR⁷—S(O)ₙA and/or carbonyl oxygen;

Hal is —F, —Cl, —Br or —I;

I is 0 or 1;

m is 1 or 2;

n is 0, 1 or 2;

and their pharmaceutically tolerated salts and solvates,

p) compounds of the formula I

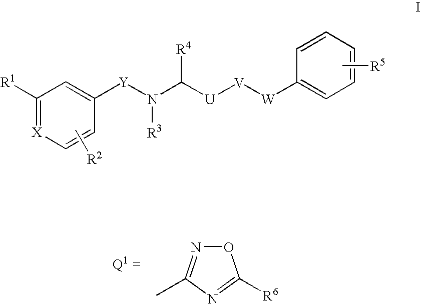

in which

R¹ is H, Cl, F, OH, OA, O—(CH₂)ₙ—Ar, NH₂, NHCOA, NHCOOA, NH—(CH₂)ₙ—Ar, CN, CONH₂, CSNH₂, C[NH]SA, C[NH]NH₂, C[NH]NHA, C[NH]NOH, C[NH]NOA, C[NH]NOCOA, C[NH]NOCOAr, C[NH]OA, C[NH]NHNH₂, C[NH]NHNHA, C[NH]NHCOOA, C[NH]NHCOA C[NH]NHCOO—(CH₂)ₘ—Ar, C[NH]NHCOO—(CH₂)ₘ-Het, NHC[NH]NH₂, NHC[NH]NHCOOA, NHC[NH]NHCOO—(CH₂)ₘ—Ar or Q1,

R² is H or one or more A, CF₃, Br, Cl, F, COA, COOH, COOA, CONH₂, CONHA, CONA₂, CH₂NH₂, CH₂NHCOA, CH₂NHCOOA, NHSO₂A, OH, OA, OCF₃, NO₂, SO₂A, SO₂NH₂ or SO₂NHA,

R³ is H, COH, COA, COCF₃, COOA or SO₂A

R⁴ is H, A, —(CH₂)ᵣ—Ar, —(CH₂)ₙ-Het, —(CH₂)ₘ—COOR⁷, —(CH₂)ₘ—CONHR⁷, —(CH₂)ₙ—S(O)ₘA, —(CH₂)ₒ—NH₂, —(CH₂)ₒ—NHCOOA, —(CH₂)ₒ—NHCOA, —(CH₂)ₒ—NHAr, —(CH₂)ₒ—NHC[NH]NH₂, —(CH₂)ₒ—(C[A]OH)-A, —(CH₂)ₒ—OH, —(CH₂)ₒ—OA, —(CH₂)ₒ—OAr, —(CH₂)ₒ-OHet, —(CH₂)ₒ—OCOOA, —(CH₂)ₒ—OCOA, —(CH₂)ₒ—OCOAr, Ar or Het,

R⁵ is —(CH₂)ₙ—COOH, —(CH₂)ₙ—COOA, —(CH₂)ₙ—COO(CH₂)ₙAr, Ar, Py or R²,

R⁶ is OH, A or Ar,

R⁷ is H, A, Ar or Het,

U is CO or CH₂,

V is NH, CO or O,

w is a bond or CO,

X is CH or N,

Y is a bond or CH₂, CO or SO₂,

n is 1 or 2,

m is 0, 1 or 2,

o is 1, 2, 3, 4 or 5,

p is 2, 3 or 4,

A is alkyl having 1-20 carbon atoms (linear, branched or cyclic), in which one or two CH₂ groups may be replaced by O or S atoms or by —CH═CH— or —C≡C— groups and in addition 1-7H atoms may be replaced by F,

Ar is phenyl or naphthyl, each of which is unsubstituted or monosubstituted, disubstituted or trisubstituted by A, CF₃, Hal, OA, OCF₃, SO₂A, SO₂NH₂, SO₂NHA, SO₂NA₂, NH₂, NHA, NA₂, NHCHO, NHCOA, NHCOOA, NACOOA, NHSO₂A, NHSO₂Ar, COOH, COOA, COO—(CH₂)ₘ—Ar, COO—(CH₂)ₘ-Het CONH₂, CONHA, CONA₂, CONHAr, COA, COAr, CH₂Ar, —(CH₂)ₘ—NH₂, —(CH₂)ₘ—NHA, —(CH₂)ₘ—NA₂, —(CH₂)ₘ—NHCHO, —(CH₂)ₘ—NHCOA, —(CH₂)ₘ—NHCOOA —(CH₂)ₘ—NHCOO—(CH₂)ₘAr, —(CH₂)ₘ—NHCOO—(CH₂)ₘ-Het, —(CH₂)ₘ-Hal, —(CH₂)ₘ-Het, NO₂, CN, CSNH₂, C[NH]SA, C[NH]OA, C[NH]NH₂, C[NH]NHOH, C[NH]NHCOOA or C[NH]NHCOOAr,

Het is a monocyclic or bicyclic, saturated, unsaturated or aromatic heterocyclic radical having from 1 to 4 N, O and/or S atoms, bonded via N or C, which may be unsubstituted or monosubstituted, disubstituted, trisubstituted or tetrasubstituted by A, CF₃, Hal, OH, OA, SO₂A, SO₂—(CH₂)ₘ—Ar, SO₂NH₂, SO₂NHA, SO₂NA₂, NH₂, NHA, NA₂, NHCHO, NHCOA, NHCOOA, NHSO₂A, NHSO₂Ar, COOH, COOA, COO—[CH₂]ₘ—Ar, CONH₂, CONHA, COA, COAr, CH₂NH₂, CH₂NHA, CH₂NHCHO, CH₂NHCOA, CH₂NHCOOA, NO₂, CN, CSNH₂, C[NH]SA, C[NH]OA, C[NH]NH₂, C[NH]NHOH, C[NH]NHCOOA, C[NH]NHCOOAr and/or carbonyl oxygen,

Py is 2-, 3- and/or 4-pyridyl, unsubstituted or monosubstituted or polysubstituted by A, Hal, CN, CONH₂, CONHA, COOH, COOA, CH₂NH₂, CH₂NHA, CH₂NHCHO, CH₂NHCOA, CH₂NHCOOA, CH₂OH, CH₂OA, CH₂OAr, CH₂OCOA, NO₂, NH₂, NHA or NA₂,

Hal is F, Cl, Br or I,

and their pharmaceutically tolerated salts and solvates,

q) compounds of the formula I

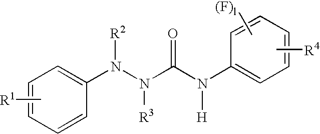

in which

R¹ is —(CH₂)ₙ—NH₂, —CON═C(NH₂)₂, —NHC(═NH)—NH₂ or —C(═NH)—NH₂, which may also be monosubstituted by —OH, —OCOOA, —OCOO(CH₂)ₙN(A)₂, —OCOO(CH₂)ₘ-Het, —CO—C(A)₂—R⁵, —COOA, —COSA, —COOAr, —COOAr′ or

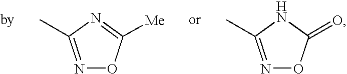

R² is H or COOA,

R³ is unbranched, branched or cyclic alkyl having 1-20 carbon atoms, in which one or two CH₂ groups may be replaced by O or S atoms, or is Ar, Ar′, X or Hal,

R⁴ is phenyl which is monosubstituted by S(O)ₖA, S(O)ₖNHA, CF₃, COOA, CH₂NHA, CN or OA,

R⁵ is —CHal₃, —O(C═O)A or

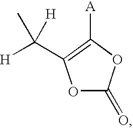

Ar is phenyl or naphthyl, each of which is unsubstituted or monosubstituted, disubstituted or trisubstituted by A, OH, OA, NH₂, NHA, NA₂, NO₂, CF₃, CN, Hal, NHCOA, COOA, CONH₂, CONHA, CONA₂, S(O)ₙA, S(O)ₙNH₂, S(O)NHA or S(O)ₙNA₂,

Ar′ is —(CH₂)ₙ—Ar,

Het is a monocyclic or bicyclic, saturated, unsaturated or aromatic heterocyclic radical having from 1 to 4 N, O and/or S atoms, bonded via N or C, which may be unsubstituted or substituted by A,

A is H or unbranched, branched or cyclic alkyl having 1-20 carbon atoms,

X is —(CH₂)ₙ—Y,

Y is COOA,

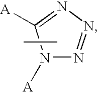

Hal is F, Cl, Br or I,

n is 1, 2, 3, 4, 5 or 6,

m is 0 or 1,

k is 0, 1 or 2,

l is 0, 1, 2, 3 or 4,

and their pharmaceutically tolerated salts and solvates,

r) compounds of the formula I

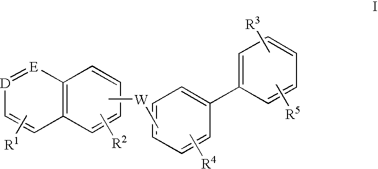

in which

-D=E- is —N═C(NH₂)— or —C(NH₂)═N—,

R¹ and R², independently of one another, are H, A, OR⁶, N(R⁶)₂, NO₂, CN, Hal, NR⁶COA, NR⁶COAr′, NR⁶SO₂A, NR⁶SO₂Ar′, COOR⁶, CON(R⁶)₂, CONR⁶Ar′, COR⁷, COAr′ or S(O)ₙA,

R³ is SO₂(NR⁶)₂, S(O)ₙA, CF₃, COOR⁶, OA or CN,

R⁴ and R⁵, independently of one another, are H, A, OR⁶, N(R⁶)₂, NO₂, CN, Hal, NR⁶COA, NR⁶COAr′, NR⁶SO₂A, NR⁶SO₂Ar′, COOR⁶, CON(R⁶)₂, CONR⁶Ar′, COR⁷, COAr′ or S(O)ₙA,

R⁶ is H, A, [C(R⁷)₂]ₙAr′ or [C(R⁷)₂]ₙHet,

R⁷ is H or A,

W is CONR⁶C(R⁶)₂CONR⁶[C(R⁶)₂]₁—, —NR⁶C(R⁶)₂CONR⁶ [C(R⁶)₂]₁—, —[C(R⁶)₂]ₘCONR⁶[C(R⁶)₂]₁— or —OC(R⁶)₂CONR⁶[C(R⁶)₂]₁—,

A is alkyl having 1-20 carbon atoms, in which one or two CH₂ groups may be replaced by O or S atoms or by —CH═CH— groups and in addition 1-7H atoms may be replaced by F,

Ar is phenyl or naphthyl, each of which is unsubstituted or monosubstituted, disubstituted or trisubstituted by A, Ar′, Het, OR⁶, N(R⁶)₂, NO₂, CN, Hal, NR⁶COA, NR⁶COAr′, NR⁶SO₂A, NR⁶SO₂Ar′, COOR⁶, CON(R⁶)₂, CONR⁶Ar′, COR⁷, COAr′, SO₂NR⁶, S(O)ₙAr′ or S(O)ₙA,

Ar′ is phenyl or naphthyl, each of which is unsubstituted or monosubstituted, disubstituted or trisubstituted by A, OR⁷, N(R⁷)₂, NO₂, CN, Hal, NR⁷COA, NR⁷SO₂A, COOR⁷, CON(R⁷)₂, COR⁷, SO₂NR⁷ or S(O)ₙA,

Het is a monocyclic or bicyclic, saturated, unsaturated or aromatic heterocyclic radical having from 1 to 4 N, O and/or S atoms, bonded via N or C, which may be unsubstituted or monosubstituted, disubstituted or trisubstituted by A, OR⁷, N(R⁷)₂, NO₂, CN, Hal, NR⁷COA, NR⁷SO₂A, COOR⁷, CON(R⁷)₂, COR⁷, SO₂NR⁷ S(O)ₙA and/or carbonyl oxygen,

Hal is F, Cl, Br or I,

n is 0, 1 or 2,

m is 1 or 2,

l is 0 or 1,

and their pharmaceutically tolerated salts and solvates,

s) compounds of the formula I

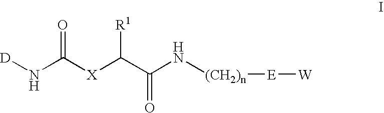

in which

D is phenyl or pyridyl, each of which is unsubstituted or monosubstituted or polysubstituted by Hal, A, OR², N(R²)₂, NO₂, CN, COOR² or CON(R²)₂,

R¹ is H, Ar, Het, cycloalkyl or A, which may be substituted by OR², SR², N(R²)₂, Ar, Het, cycloalkyl, CN, COOR² or CON(R²)₂,

R² is H or A,

E is phenylene, which may be monosubstituted or polysubstituted by Hal, A, OR², N(R²)₂, NO₂, CN, COOR² or CON(R²)₂,

or is piperidine-1,4-diyl,

W is Ar, Het or N(R²)₂

and, if E=piperidine-1,4-diyl, is alternatively R² or cycloalkyl,

X is NH or O,

A is unbranched or branched alkyl having 1-10 carbon atoms, in which one or two CH₂ groups may be replaced by O or S atoms and/or by —CH═CH— groups and/or in addition 1-7H atoms may be replaced by F,

Ar is phenyl which is unsubstituted or monosubstituted, disubstituted or trisubstituted by Hal, A, OR², N(R²)₂, NO₂, CN, COOR², CON(R², NR²COA, NR²SO₂A, COR², SO₂NR, SO₃H or S(O)ₘA,

Het is a monocyclic or bicyclic, saturated, unsaturated or aromatic heterocyclic radical having from 1 to 4 N, O and/or S atoms, which may be unsubstituted or monosubstituted, disubstituted or trisubstituted by Hal, A, OR², N(R²)₂, NO₂, CN, COOR², CON(R²)₂, NR²COA, NR²SO₂A, COR², SO₂NR², SO₃H or S(O)ₘA and/or carbonyl oxygen,

Hal is F, Cl, Br or I,

n is 0 or 1,

m is 0, 1 or 2,

and their pharmaceutically tolerated salts and solvates.

Other preferred factor Xa inhibitors are, for example, the compounds described in the following documents:

a) in WO 97/30971, page 4, line 5, to page 13, line 19;

b) in EP 0 921 116 A1, page 2, line 1, to line 51;

c) in EP 0 540 051 B1, page 2, line 41, to page 3, line 14;

d) in EP 0 798 295 A1, page 69, line 10, to page 71, page 53;

Other preferred compounds are selected from the group consisting of defibrotide, desirudin and lepirudin.

The invention preferably relates to a formulation comprising [7-(3-chloro-4-methoxybenzylamino)-1-methyl-3-propyl-1H-pyrazolo[4,3-d]-pyrimidin-5-ylmethoxy]acetic acid and physiologically acceptable salts and/or solvates thereof and a calcium antagonist.

Besides the free acid, the ethanolamine salt is preferred.

Preference is given to calcium antagonists selected from the group consisting of selective and non-selective calcium antagonists.

Preference is given to selective calcium antagonists selected from the group consisting of dihydropyridine derivatives, phenylalkylamine derivatives, benzothiazepine derivatives and other selective calcium antagonists.

Dihydropyridine derivatives are preferably selected from the group consisting of amlodipine, felodipine, isradipine, nicardipine, nifedipine, nimodipine, nisoldipine, nitrendipine, lacidipine, nilvadipine, manidipine, bamidipine and lercanidipine.

The phenylalkylamine derivatives are preferably selected from the group consisting of verapamil and gallopamil.

The benzothiazepine derivatives are preferably diltiazem.

The other selective calcium antagonists are preferably mibefradil.

The non-selective calcium antagonists are preferably selected from the group consisting of fendiline, bepridil, lidoflazine and perhexiline.

The invention preferably relates to a formulation comprising [7-(3-chloro-4-methoxybenzylamino)-1-methyl-3-propyl-1H-pyrazolo[4,3-d]-pyrimidin-5-ylmethoxy]acetic acid and physiologically acceptable salts and/or solvates thereof and a prostaglandin or prostaglandin derivative. Besides the free acid, the ethanolamine salt is preferred.

Preference is given to prostaglandins or prostaglandin derivatives selected from the group consisting of PGE₀, PGA₁, PGB₁, PGF_{1α}, PGA₂, PGB₂, 19-hydroxy-PGA₁, 19-hydroxy-PGB₁, 19-hydroxy-PGA₂, 19-hydroxy-PGB₂, PGE₃, PGF_{3α}, alprostadil (PGE₁), dinoprost (PGF₂), dinoprostone (PGE₂), epoprostenol sodium (PGI₂; prostacyclin sodium), gemeprost, iloprost, latanoprost, misoprostol, sulprostone, carboprost thromethamin, dinoprost thromethamin, lipoprost, metenoprost and tiaprost.

Particular preference is given to prostaglandins or prostaglandin derivatives selected from the group consisting of alprostadil (PGE₁), dinoprost (PGF₂), dinoprostone (PGE₂), epoprostenol sodium (PGI₂; prostacyclin sodium), gemeprost, iloprost, latanoprost, misoprostol, sulprostone, carboprost thromethamin, dinoprost thromethamin, lipoprost, metenoprost and tiaprost.

Particular preference is given to PGE₁ or prostacyclin, especially preferably prostacyclin.

The compounds of the formula I and also the starting materials for their preparation are, in addition, prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants which are known per se, but are not mentioned here in greater detail.

In the compounds of the formula II or III, R¹, R², R³, R⁴ and X have the meanings indicated, in particular the preferred meanings indicated.

If L is a reactive esterified OH group, this is preferably alkylsulfonyloxy having 1-6 carbon atoms (preferably methylsulfonyloxy) or arylsulfonyloxy having 6-10 carbon atoms (preferably phenyl- or p-tolylsulfonyloxy, further-more also 2-naphthalenesulfonyloxy).

The compounds of the formula I can preferably be obtained by reacting compounds of the formula II with compounds of the formula III.

If desired, the starting materials can also be formed in situ by not isolating them from the reaction mixture, but instead immediately converting them further into the compounds of the formula I.

On the other hand, it is possible to carry out the reaction stepwise.

The starting compounds of the formulae II and III are generally known. If they are not known, they can be prepared by methods known per se. Compounds of the formula II can be prepared by methods known from the literature, for example from 4-amino-3-alkoxycarbonylpyrazoles by cyclisation using nitrites and subsequent reaction of the cyclisation products with phosphorus oxychloride (analogously to Houben Weyl E9b/2).

In detail, the reaction of the compounds of the formula II with the compounds of the formula III is carried out in the presence or absence of an inert solvent at temperatures between about −20 and about 1500, preferably between 20 and 100°.

The addition of an acid-binding agent, for example an alkali or alkaline earth metal hydroxide, carbonate or bicarbonate or another salt of a weak acid of the alkali or alkaline earth metals, preferably of potassium, sodium or calcium, or the addition of an organic base, such as triethylamine, dimethylamine, pyridine or quinoline or of an excess of the amine component, may be favourable.

Examples of suitable inert solvents are hydrocarbons, such as hexane, petroleum ether, benzene, toluene or xylene; chlorinated hydrocarbons, such as trichloroethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, N-methylpyrrolidone or dimethylformamide (DMF); nitriles, such as acetonitrile; sulfoxides, such as dimethyl sulfoxide (DMSO); nitro compounds, such as nitromethane or nitrobenzene; esters, such as ethyl acetate, or mixtures of the said solvents.

It is furthermore possible to convert a radical X in a compound of the formula I into another radical X, for example by hydrolysing an ester or a cyano group to give a COOH group.

Ester groups can be saponified, for example, using NaOH or KOH in water, water/THF or water/dioxane at temperatures between 0 and 100°. Carboxylic acids can be converted into the corresponding carboxylic acid chlorides, for example using thionyl chloride, and these can be converted into carboxamides. Elimination of water therefrom in a known manner gives carbonitriles.

An acid of the formula I can be converted into the associated acid-addition salt using a base, for example by reaction of equivalent amounts of the acid and the base in an inert solvent, such as ethanol, followed by evaporation. Suitable bases for this reaction are, in particular, those which give physiologically acceptable salts.

Thus, the acid of the formula I can be converted into the corresponding metal salt, in particular alkali metal or alkaline earth metal salt, or into the corresponding ammonium salt using a base (for example sodium hydroxide, potassium hydroxide, sodium carbonate or potassium carbonate). Also suitable for this reaction are, in particular, organic bases which give physiologically acceptable salts, such as, for example, ethanolamine.

On the other hand, a base of the formula I can be converted into the associated acid-addition salt using an acid, for example by reaction of equivalent amounts of the base and the acid in an inert solvent, such as ethanol, followed by evaporation. Suitable acids for this reaction are, in particular, those which give physiologically acceptable acids. Thus, it is possible to use inorganic acids, for example sulfuric acid, nitric acid, hydrohalic acids, such as hydrochloric acid or hydrobromic acid, phosphoric acids, such as orthophosphoric acid, or sulfamic acid, furthermore organic acids, in particular aliphatic, alicyclic, araliphatic, aromatic or heterocyclic monobasic or polybasic carboxylic, sulfonic or sulfuric acids, for example formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, p-toluenesulfonic acid, naphthalenemono- and -disulfonic acids, or laurylsulfuric acid. Salts with physiologically unacceptable acids, for example picrates, can be used for the isolation and/or purification of the compounds of the formula I.

The invention furthermore relates to pharmaceutical formulations comprising at least one compound of the formula I and/or one of its physiologically acceptable salts and at least one antithrombotic, a calcium antagonist or at least one prostaglandin or prostaglandin derivative, and comprising one or more excipients and/or assistants.

The pharmaceutical preparations are prepared, in particular, by non-chemical methods. The active ingredients are converted into a suitable dosage form here together with at least one solid, liquid and/or semi-liquid excipient or assistant.

These preparations can be used as medicaments in human or veterinary medicine. Suitable excipients are organic or inorganic substances which are suitable for enteral (for example oral), parenteral or topical administration and do no react with the novel compounds, for example water, vegetable oils, benzyl alcohols, alkylene glycols, polyethylene glycols, glycerol triacetate, gelatine, carbohydrates, such as lactose or starch, magnesium stearates, talc or vaseline. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, and suitable for topical application are ointments, creams or powders. The novel compounds may also be lyophilised and the resultant lyophilisates used, for example, for the preparation of injection preparations. The preparations indicated may be sterilised and/or comprise assistants, such as lubricants, preservatives, stabilisers and/or wetting agents, emulsifiers, salts for modifying the osmotic pressure, buffer substances, colorants and flavours and/or a plurality of further active ingredients, for example one or more vitamins. They can furthermore be administered as nasal sprays.

In general, the substances are preferably administered in doses of between about 1 and 500 mg, in particular between 5 and 100 mg per dosage unit. The daily dose is preferably between about 0.02 and 10 mg/kg of body weight. However, the specific dose for each patient depends on a wide variety of factors, for example on the efficacy of the specific compound employed, on the age, body weight, general state of health, sex, on the diet, on the time and method of administration, on the excretion rate, medicament combination and severity of the particular illness to which the therapy applies. Oral administration is preferred.

The invention therefore also relates to the use of the pharmaceutical preparations described for the preparation of a medicament for the treatment of angina, high blood pressure, pulmonary hypertension, congestive heart failure (CHF), chronic obstructive pulmonary disease (COPD), cor pulmonale, dextrocardiac insufficiency, atherosclerosis, conditions of reduced patency of heart vessels, peripheral vascular diseases, strokes, bronchitis, allergic asthma, chronic asthma, allergic rhinitis, glaucoma, irritable bowel syndrome, tumours, renal insufficiency, liver cirrhosis and for the treatment of female sexual disorders.

The invention relates in particular to the use of the formulations according to the invention for the preparation of a medicament for the treatment of pulmonary hypertension, congestive heart failure (CHF), chronic obstructive pulmonary disease (COPD), cor pulmonale and/or dextrocardiac insufficiency.

The constituents of the novel pharmaceutical preparation are preferably administered in combination. However, they can also be administered individually at the same time or successively.

The invention also relates to a set (kit) consisting of separate packs of

(a) an effective amount of [7-(3-chloro-4-methoxybenzylamino)-1-methyl-3-propyl-1H-pyrazolo[4,3-d]pyrimidin-5-ylmethoxy]acetic acid, ethanolamine salt, and

(b) an effective amount of an antithrombotic.

The set comprises suitable containers, such as boxes, individual bottles, bags or ampoules. The set may, for example, comprise separate ampoules each containing an effective amount of [7-(3-chloro-4-methoxy-benzylamino)-1-methyl-3-propyl-1H-pyrazolo[4,3-d]pyrimidin-5-ylmethoxy]acetic acid, ethanolamine salt, and of the antithrombotic in dissolved or lyophilised form.

The invention furthermore relates to the use of [7-(3-chloro-4-methoxy-benzylamino)-1-methyl-3-propyl-1H-pyrazolo[4,3-d]pyrimidin-5-ylmethoxy]acetic acid, ethanolamine salt, for the preparation of a medicament for the treatment of pulmonary hypertension, congestive heart failure (CHF), chronic obstructive pulmonary disease (COPD), cor pulmonale and/or dextrocardiac insufficiency

The invention also relates to a set (kit) consisting of separate packs of

(a) an effective amount of [7-(3-chloro-4-methoxybenzylamino)-1-methyl-3-propyl-1H-pyrazolo[4,3-d]pyrimidin-5-ylmethoxy]acetic acid, ethanolamine salt, and

(b) an effective amount of a calcium antagonist.

The set comprises suitable containers, such as boxes, individual bottles, bags or ampoules. The set may, for example, comprise separate ampoules each containing an effective amount of of [7-(3-chloro-4-methoxybenzylamino)1-methyl-3-propyl-1H-pyrazolo[4,3-d]pyrimidin-5-ylmethoxy]acetic acid, ethanolamine salt, and of the calcium antagonist in dissolved or lyophilised form.

The invention also relates to a set (kit) consisting of separate packs of

(a) an effective amount of [7-(3-chloro-4-methoxybenzylamino)-1-methyl-3-propyl-1H-pyrazolo[4,3-d]pyrimidin-5-ylmethoxy]acetic acid, ethanolamine salt, and

(b) an effective amount of a prostaglandin or prostaglandin derivative.

The set comprises suitable containers, such as boxes, individual bottles, bags or ampoules. The set may, for example, comprise separate ampoules each containing an effective amount of of [7-(3-chloro-4-methoxybenzylamino)-1-methyl-3-propyl-1H-pyrazolo[4,3-d]pyrimidin-5-ylmethoxy]acetic acid, ethanolamine salt, and of the prostaglandin or prostaglandin derivative in dissolved or lyophilised form.

Above and below, all temperatures are given in ° C. In the following examples, “conventional work-up” means that water is added if necessary, a pH of between 2 and 10, depending on the constitution of the final product, is established if necessary, the mixture is extracted with ethyl acetate or dichloromethane, the phases are separated, the organic phase is dried over sodium sulfate and evaporated, and the product is purified by chromatography on silica gel and/or by crystallisation.

Mass spectrometry (MS): El (electron impact ionisation) M⁺

FAB (fast atom bombardment) (M+H)⁺

### EXAMPLE 1

3 g of methyl 3-[7-chloro-1-methyl-3-propyl-1H-pyrazolo[4,3-d]pyrimidin-5-yl]propionate and 1.9 g of 3-chloro-4-methoxybenzylamine (“A”) in 50 ml of dimethylformamide (DMF) are stirred at 60° for 12 hours in the presence of potassium carbonate. After filtration, the solvent is removed, and the mixture is subjected to conventional work-up, giving 4.6 g of methyl 3-[7-(3-chloro-4-methoxybenzylamino)-1-methyl-3-propyl-1H-pyrazolo[4,3-d]-pyrimidin-5-yl]propionate as a colourless oil.

Analogous Reaction of “A”

with methyl 2-[7-chloro-1-methyl-3-propyl-1H-pyrazolo[4,3-d]pyrimidin-5-yl]-acetate gives

methyl 2-[7-(3-chloro-4-methoxybenzylamino)-1-methyl-3-propyl-1H-pyrazolo[4,3-d]pyrimidin-5-yl]acetate.

Analogous Reaction of 3,4-methylenedioxybenzylamine

with methyl 3-[7-chloro-1-methyl-3-propyl-1H-pyrazolo[4,3-d]pyrimidin-5-yl]propionate gives

methyl 3-[7-(3,4-methylenedioxybenzylamino)-1-methyl-3-propyl-1H-pyrazolo[4,3-d]pyrimidin-5-yl]propionate.

Analogous Reaction of “A”

with methyl 4-[7-chloro-1-methyl-3-propyl-1H-pyrazolo[4,3-d]pyrimidin-5-yl]butyrate gives

methyl 4-[7-(3-chloro-4-methoxybenzylamino)-1-methyl-3-propyl-1H-pyrazolo[4,3-d]pyrimidin-5-yl]butyrate.

Analogous Reaction of 3,4-methylenedioxybenzylamine

with methyl 4-[7-chloro-1-methyl-3-propyl-1H-pyrazolo[4,3-d]pyrimidin-5-yl]butyrate gives

methyl 4-[7-(3,4-methylenedioxybenzylamino)-1-methyl-3-propyl-1H-pyrazolo[4,3-d]pyrimidin-5-yl]butyrate.

Analogous Reaction of “A”

with methyl 5-[7-chloro-1-methyl-3-propyl-1H-pyrazolo[4,3-d]pyrimidin-5-yl]valerate gives

methyl 5-[7-(3-chloro-4-methoxybenzylamino)-1-methyl-3-propyl-1H-pyrazolo[4,3-d]pyrimidin-5-yl]valerate.

Analogous Reaction of 3,4-methylenedioxybenzylamine

with methyl 5-[7-chloro-1-methyl-3-propyl-1H-pyrazolo[4,3-d]pyrimidin-5-yl]valerate gives

methyl 5-[7-(3,4-methylenedioxybenzylamino)-1-methyl-3-propyl-1H-pyrazolo[4,3-d]pyrimidin-5-yl]valerate.

Analogous Reaction of “A”

with methyl 7-[7-chloro-1-methyl-3-propyl-1H-pyrazolo[4,3-d]pyrimidin-5-yl]heptanoate gives

methyl 7-[7-(3-chloro-4-methoxybenzylamino)-1-methyl-3-propyl-1H-pyrazolo[4,3-d]pyrimidin-5-yl]heptanoate.

Analogous reaction of 3,4-methylenedioxybenzylamine with methyl 7-[7-chloro-1-methyl-3-propyl-1H-pyrazolo[4,3-d]pyrimidin-5-yl]heptanoate gives

methyl 7-[7-(3,4-methylenedioxybenzylamino)-1-methyl-3-propyl-1H-pyrazolo[4,3-d]pyrimidin-5-yl]heptanoate.

Analogous Reaction of “A”

with methyl 2-[4-(7-chloro-1-methyl-3-propyl-1H-pyrazolo[4,3-d]-pyrimidin-5-yl)-cyclohex-1-yl]acetate gives

methyl 2-{4-[7-(3-chloro-4-methoxybenzylamino)-1-methyl-3-propyl-1H-pyrazolo[4,3-d]pyrimidin-5-yl]-cyclohexyl-1-yl}acetate.

Analogous Reaction of 3,4-methylenedioxybenzylamine

with methyl 2-[4-(7-chloro-1-methyl-3-propyl-1H-pyrazolo[4,3-d]-pyrimidin-5-yl)-cyclohex-1-yl]acetate gives

methyl 2-{4-[7-(3,4-methylenedioxybenzylamino)-1-methyl-3-propyl-1H-pyrazolo[4,3-d]pyrimidin-5-yl]-cyclohexyl-1-yl}acetate.

Analogous Reaction of Benzylamine

with methyl 3-[7-chloro-1-methyl-3-propyl-1H-pyrazolo[4,3-d]pyrimidin-5-yl]propionate gives

methyl 3-[7-benzylamino-1-methyl-3-propyl-1H-pyrazolo[4,3-d]-pyrimidin-5-yl]propionate;

with methyl 4-[7-chloro-1-methyl-3-propyl-1H-pyrazolo[4,3-d]pyrimidin-5-yl]butyrate gives

methyl 4-[7benzylamino-1-methyl-3-propyl-1H-pyrazolo[4,3-d]-pyrimidin-5-yl]butyrate;

with methyl 5-[7-chloro-1-methyl-3-propyl-1H-pyrazolo[4,3-d]pyrimidin-5-yl]valerate gives

methyl 5-[7benzylamino-1-methyl-3-propyl-1H-pyrazolo[4,3-d]-pyrimidin-5-yl]valerate.

Analogous Reaction of “A”

with methyl 4-[7-chloro-1-methyl-3-propyl-1H-pyrazolo[4,3-d]pyrimidin-5-yl]cyclohexanecarboxylate gives

methyl 4-[7-(3-chloro-4-methoxybenzylamino)-1-methyl-3-propyl-1H-pyrazolo[4,3-d]pyrimidin-5-yl]cyclohexanecarboxylate and reaction of 3,4-methylenedioxybenzylamine gives

methyl 4-[7-(3,4-methylendioxybenzylamino)-1-methyl-3-propyl-1H-pyrazolo[4,3-d]pyrimidin-5-yl]cyclohexanecarboxylate.

### EXAMPLE 2

4.3 g of methyl 3-[7-(3-chloro-4-methoxybenzylamino)-1-methyl-3-propyl-1H-pyrazolo[4,3-d]pyrimidin-5-yl]propionate are dissolved in 30 ml of tetrahydrofuran (THF), 10 ml of 10% NaOH are added, and the mixture is stirred at 60° for 8 hours. After 10% HCl has been added, the deposited crystals are separated off and recrystallised from methanol, giving 3.7 g of 3-[7-(3-chloro-4-methoxybenzylamino)-1-methyl-3-propyl-1H-pyrazolo-[4,3-d]pyrimidin-5-yl]propionic acid, m.p. 178°.

Evaporation with the equivalent amount of methanolic potassium hydroxide solution gives the potassium salt of the acid as an amorphous powder.

Analogous reaction of the esters listed in Example 1 gives the compounds

2-[7-(3-chloro-4-methoxybenzylamino)-1-methyl-3-propyl-1H-pyrazolo[4,3-d]pyrimidin-5-yl]acetic acid,

3-[7-(3,4-methylenedioxybenzylamino)-1-methyl-3-propyl-1H-pyrazolo[4,3-d]pyrimidin-5-yl]propionic acid,

4-[7-(3-chloro-4-methoxybenzylamino)-1-methyl-3-propyl-1H-pyrazolo[4,3-d]pyrimidin-5-yl]butyric acid, m.p. 152°;

4-[7-(3,4-methylenedioxybenzylamino)-1-methyl-3-propyl-1H-pyrazolo[4,3-d]pyrimidin-5-yl]butyric acid, m.p. 172°;

5-[7-(3-chloro-4-methoxybenzylamino)-1-methyl-3-propyl-1H-pyrazolo[4,3-d]pyrimidin-5-yl]valeric acid, m.p. 159°;

5-[7-(3,4-methylenedioxybenzylamino)1-methyl-3-propyl-1H-pyrazolo[4,3-d]pyrimidin-5-yl]valeric acid, ethanolamine salt, m.p. 160°;

7-[7-(3-chloro-4-methoxybenzylamino)-1-methyl-3-propyl-1H-pyrazolo[4,3-d]pyrimidin-5-yl]heptanoic acid,

7-[7-(3,4-methylenedioxybenzylamino)-1-methyl-3-propyl-1H-pyrazolo[4,3-d]pyrimidin-5-yl]heptanoic acid,

2-{4-[7-(3-chloro-4-methoxybenzylamino)-1-methyl-3-propyl-1H-pyrazolo[4,3-d]pyrimidin-5-yl]cyclohexyl-1-yl}acetic acid,

2-{4-[7-(3,4-methylenedioxybenzylamino)-1-methyl-3-propyl-1H-pyrazolo[4,3-d]pyrimidin-5-yl]cyclohexyl-1-yl}acetic acid,

3-[7benzylamino-1-methyl-3-propyl-1H-pyrazolo[4,3-d]pyrimidin-5-yl]-propionic acid,

4-[7benzylamino-1-methyl-3-propyl-1H-pyrazolo[4,3-d]pyrimidin-5-yl]-butyric acid,

5-[7benzylamino-1-methyl-3-propyl-1H-pyrazolo[4,3-d]pyrimidin-5-yl]-valeric acid, m.p. 185°;

4-[7-(3-chloro-4-methoxybenzylamino)-1-methyl-3-propyl-1H-pyrazolo[4,3-d]pyrimidin-5-yl]cyclohexanecarboxylic acid,

4-[7-(3,4-methylenedioxybenzylamino)-1-methyl-3-propyl-1H-pyrazolo[4,3-d]pyrimidin-5-yl]cyclohexanecarboxylic acid.

An analogous reaction gives the compounds

5-[7-(3-chloro-4-methoxybenzylamino)-1-methyl-3-isopropyl-1H-pyrazolo[4,3-d]pyrimidin-5-yl]valeric acid, cyclohexylamine salt, m.p. 148°;

4-[7-(3-chloro-4-methoxybenzylamino)-1-methyl-3-ethyl-1H-pyrazolo[4,3-d]pyrimidin-5-yl]butyric acid, m.p. 176°;

4-[7-(3,4-methylenedioxybenzylamino)-1-methyl-3-ethyl-1H-pyrazolo[4,3-d]pyrimidin-5-yl]butyric acid, m.p. 187°;

4-[7-(3-chloro-4-methoxybenzylamino)-1-ethyl-3-methyl-1H-pyrazolo[4,3-d]pyrimidin-5-yl]butyric acid, m.p. 206°;

4-[7-(3,4-methylenedioxybenzylamino)-1-ethyl-3-methyl-1H-pyrazolo[4,3-d]pyrimidin-5-yl]butyric acid, m.p. 177°;

4-[7benzylamino-1-methyl-3-ethyl-1H-pyrazolo[4,3-d]pyrimidin-5-yl]-butyric acid, m.p. 208°;

4-[7-(3-chloro-4-methoxybenzylamino)-1-methyl-3-methyl-1H-pyrazolo[4,3-d]pyrimidin-5-yl]butyric acid, m.p. 250°;

4-[7-(3,4-methylenedioxybenzylamino)-1-methyl-3-methyl-1H-pyrazolo[4,3-d]pyrimidin-5-yl]butyric acid, m.p. 225°;

4-[7benzylamino-1-methyl-3-methyl-1H-pyrazolo[4,3-d]pyrimidin-5-yl]-butyric acid, m.p. 201°;

5-[7-(4-Methoxybenzylamino)-1-methyl-3-propyl-1H-pyrazolo[4,3-d]-pyrimidin-5-yl]valeric acid, m.p. 160°;

5-[7-(3-Methoxybenzylamino)-1-methyl-3-propyl-1H-pyrazolo[4,3-d]-pyrimidin-5-yl]valeric acid, m.p. 141°;

5-[7-(4-chlorobenzylamino)-1-methyl-3-propyl-1H-pyrazolo[4,3-d]-pyrimidin-5-yl]valeric acid, m.p. 148°;

5-[7-(3-chlorobenzylamino)-1-methyl-3-propyl-1H-pyrazolo[4,3-d]-pyrimidin-5-yl]valeric acid, m.p. 151°;

### EXAMPLE 3

A mixture of 1.8 g of methyl 4-[7-chloro-1-methyl-3-propyl-1H-pyrazolo-[4,3-d]pyrimidin-5-yl]phenylcarboxylate (“B”) and 1.5 g of 3-chloro-4-methoxybenzylamine in 20 ml of N-methylpyrrolidone is heated at 110° for 4 hours. After cooling, the mixture is subjected to conventional work-up, giving 2.2 g of methyl 4-[7-(3-chloro-4-methoxybenzylamino1-methyl-3-propyl-1H-pyrazolo[4,3-d]pyrimidin-5-yl]benzoate.

Analogously to Example 2, 1.2 g of the ester give 1.0 g of

4-[7-(3-chloro-4-methoxybenzylamino1-methyl-3-propyl-1H-pyrazolo[4,3-d]pyrimidin-5-yl]benzoic acid, ethanolamine salt, m.p. 139°.

Analogously to Example 1, “B” and 3,4-methylenedioxybenzylamine give

methyl 4-[7-(3,4-methylenedioxybenzylamino)-1-methyl-3-propyl-1H-pyrazolo[4,3-d]pyrimidin-5-yl]benzoate, and ester hydrolysis thereof gives

4-[7-(3,4-methylenedioxybenzylamino)-1-methyl-3-propyl-1H-pyrazolo[4,3-d]pyrimidin-5-yl]benzoic acid.

An analogous reaction gives the compound

4-[7-(3-chloro-4-methoxybenzylamino)-1-methyl-3-propyl-1H-pyrazolo[4,3-d]pyrimidin-5-yl]-phenylessigsäure, glucamine salt, m.p. 114° and

4-[7-(3,4-methylenedioxybenzylamino)-1-methyl-3-propyl-1H-pyrazolo[4,3-d]pyrimidin-5-yl]phenyl acetic acid.

### EXAMPLE 4

1 equivalent of 3-[7-(3-chloro-4-methoxybenzylamino)-1-methyl-3-propyl-1H-pyrazolo[4,3-d]pyrimidin-5-yl]propionic acid and 1.2 equivalents of thionyl chloride are stirred in dichloromethane for 2 hours. The solvent is removed, giving 3-[7-(3-chloro-4-methoxybenzylamino)-1-methyl-3-propyl-1H-pyrazolo[4,3-d]pyrimidin-5-yl]propionyl chloride.

The product is transferred into aqueous ammonia, and the mixture is stirred for one hour and subjected to conventional work-up, giving 3-[7-(3-chloro-4-methoxybenzylamino)-1-methyl-3-propyl-1H-pyrazolo[4,3-d]-pyrimidin-5-yl]propionamide.

### EXAMPLE 5

1 equivalent of DMF and 1 equivalent of oxalyl chloride are dissolved in acetonitrile at 0°. 1 equivalent of 3-[7-(3-chloro-4-methoxybenzylamino)-1-methyl-3-propyl-1 H-pyrazolo[4,3-d]pyrimidin-5-yl]propionamide is then added. The mixture is stirred for one hour. Conventional work-up gives 3-[7-(3-chloro-4-methoxybenzylamino)-1-methyl-3-propyl-1H-pyrazolo[4,3-d]-pyrimidin-5-yl]-propionitrile.

### EXAMPLE 6

Reaction of the corresponding chloro-pyrimidine derivates with 3,4-ethylenedioxybenzylamine analogously to Examples 1, 2 and 3 gives the following carboxylic acids

4-[7-(3,4-ethylenedioxybenzylamino)-1-methyl-3-propyl-1H-pyrazolo-[4,3-d]pyrimidin-5-yl]butyric acid,

3-[7-(3,4-ethylenedioxybenzylamino)-1-methyl-3-propyl-1H-pyrazolo-[4,3-d]pyrimidin-5-yl]propionic acid,

5-[7-(3,4-ethylenedioxybenzylamino)-1-methyl-3-propyl-1H-pyrazolo-[4,3-d]pyrimidin-5-yl]valeric acid,

7-[7-(3,4-ethylenedioxybenzylamino)-1-methyl-3-propyl-1H-pyrazolo-[4,3-d]pyrimidin-5-yl]heptanoic acid,

2-{4-[7-(3,4-ethylenedioxybenzylamino)-1-methyl-3-propyl-1H-pyrazolo[4,3-d]pyrimidin-5-yl]cyclohexyl-1-yl}acetic acid,

4-[7-(3,4-ethylenedioxybenzylamino)-1-methyl-3-propyl-1H-pyrazolo-[4,3-d]pyrimidin-5-yl]cyclohexanecarboxylic acid,

4-[7-(3,4-ethylenedioxybenzylamino)-1-methyl-3-propyl-1H-pyrazolo-[4,3-d]pyrimidin-5-yl]benzoic acid,

4-[7-(3,4-ethylenedioxybenzylamino)-1-methyl-3-propyl-1H-pyrazolo-[4,3-d]pyrimidin-5-yl]benzoic acid,

4-[7-(3,4-ethylenedioxybenzylamino)-1-methyl-3-propyl-1H-pyrazolo-[4,3-d]pyrimidin-5-yl]phenylacetic acid.

Analogous reaction with 3,4-dichlorobenzylamine gives the following compounds

4-[7-(3,4-dichlorobenzylamino)-1-methyl-3-propyl-1H-pyrazolo[4,3-d]-pyrimidin-5-yl]butyric acid, m.p. 209°;

3-[7-(3,4-dichlorobenzylamino)-1-methyl-3-propyl-1H-pyrazolo[4,3-d]-pyrimidin-5-yl]propionic acid,

5-[7-(3,4-dichlorobenzylamino)-1-methyl-3-propyl-1H-pyrazolo[4,3-d]-pyrimidin-5-yl]valeric acid,

7-[7-(3,4-dichlorobenzylamino)-1-methyl-3-propyl-1H-pyrazolo[4,3-d]-pyrimidin-5-yl]heptanoic acid,

2-{4-[7-(3,4-dichlorobenzylamino)-1-methyl-3-propyl-1H-pyrazolo-[4,3-d]pyrimidin-5-yl]cyclohexyl-1-yl}acetic acid,

4-[7-(3,4-dichlorobenzylamino)-1-methyl-3-propyl-1H-pyrazolo[4,3-d]-pyrimidin-5-yl]cyclohexanecarboxylic acid,

4-[7-(3,4-dichlorobenzylamino)-1-methyl-3-propyl-1H-pyrazolo[4,3-d]-pyrimidin-5-yl]benzoic acid,

4-[7-(3,4-dichlorobenzylamino)-1-methyl-3-propyl-1H-pyrazolo[4,3-d]-pyrimidin-5-yl]phenylacetic acid.

Analogous reaction with 3-chloro-4-ethoxybenzylamine gives the following compounds

4-[7-(3-chloro-4-ethoxybenzylamino)-1-methyl-3-propyl-1H-pyrazolo-[4,3-d]pyrimidin-5-yl]butyric acid,

3-[7-(3-chloro-4-ethoxybenzylamino)-1-methyl-3-propyl-1H-pyrazolo-[4,3-d]pyrimidin-5-yl]propionic acid,

5-[7-(3-chloro-4-ethoxybenzylamino)-1-methyl-3-propyl-1H-pyrazolo-[4,3-d]pyrimidin-5-yl]valeric acid,

7-[7-(3-chloro-4-ethoxybenzylamino)-1-methyl-3-propyl-1H-pyrazolo-[4,3-d]pyrimidin-5-yl]heptanoic acid,

2-{4-[7-(3-chloro-4-ethoxybenzylamino)-1-methyl-3-propyl-1H-pyrazolo[4,3-d]pyrimidin-5-yl]cyclohexyl-1-yl}acetic acid,

4-[7-(3-chloro-4-ethoxybenzylamino)-1-methyl-3-propyl-1H-pyrazolo-[4,3-d]pyrimidin-5-yl]cyclohexanecarboxylic acid,

4-[7-(3-chloro-4-ethoxybenzylamino)-1-methyl-3-propyl-1H-pyrazolo-[4,3-d]pyrimidin-5-yl]benzoic acid,

4-[7-(3-chloro-4-ethoxybenzylamino)-1-methyl-3-propyl-1H-pyrazolo[4,3-d]pyrimidin-5-yl]phenylacetic acid.

Analogous reaction with 3-chloro-4-isopropoxybenzylamine gives the following compounds

4-[7-(3-chloro-4-isopropoxybenzylamino)-1-methyl-3-propyl-1H-pyrazolo[4,3-d]pyrimidin-5-yl]butyric acid,

3-[7-(3-chloro-4-isopropoxybenzylamino)-1-methyl-3-propyl-1H-pyrazolo[4,3-d]pyrimidin-5-yl]propionic acid,

5-[7-(3-chloro-4-isopropoxybenzylamino)-1-methyl-3-propyl-1H-pyrazolo[4,3-d]pyrimidin-5-yl]valeric acid,

7-[7-(3-chloro-4-isopropoxybenzylamino)-1-methyl-3-propyl-1H-pyrazolo[4,3-d]pyrimidin-5-yl]heptanoic acid,

2-{4-[7-(3-chloro-4-isopropoxybenzylamino)-1-methyl-3-propyl-1H-pyrazolo[4,3-d]pyrimidin-5-yl]cyclohexyl-1-yl}acetic acid,

4-[7-(3-chloro-4-isopropoxybenzylamino)-1-methyl-3-propyl-1H-pyrazolo[4,3-d]pyrimidin-5-yl]cyclohexanecarboxylic acid,

4-[7-(3-chloro-4-isopropoxybenzylamino)-1-methyl-3-propyl-1H-pyrazolo[4,3-d]pyrimidin-5-yl]benzoic acid,

4-[7.-(3-chloro-4-isopropoxybenzylamino)-1-methyl-3-propyl-1H-pyrazolo[4,3-d]pyrimidin-5-yl]phenylacetic acid.

### EXAMPLE 7

An analogous reaction to Examples 1 and 2 gives the compound

[7-(3-chloro-4-methoxybenzylamino)-1-methyl-3-propyl-1H-pyrazolo[4,3-d]-pyrimidin-5-ylmethoxy]acetic acid, ethanolamine salt, m.p. 138°.

The examples below relate to pharmaceutical preparations:

### EXAMPLE A: INJECTION VIALS

A solution of 100 g of an active ingredient of the formula I, 100 g of the antithrombotic and 5 g of disodium hydrogenphosphate in 3 l of bidistilled water is adjusted to pH 6.5 using 2N hydrochloric acid, sterile filtered, transferred into injection vials, lyophilised under sterile conditions and sealed under sterile conditions. Each injection vial contains 5 mg of each active ingredient.

### EXAMPLE B: SUPPOSITORIES

A mixture of 20 g of an active ingredient of the formula I, 20 g of an antithrombotic with 100 g of soya lecithin and 1400 g of cocoa butter is melted, poured into moulds and allowed to cool. Each suppository contains 20 mg of each active ingredient.

### EXAMPLE C: SOLUTION

A solution is prepared from 1 g of an active ingredient of the formula I, 1 g of an antithrombotic, 9.38 g of NaH₂PO₄.2H₂O, 28.48 g of Na₂HPO₄.12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE D: OINTMENT

500 mg of an active ingredient of the formula I and 500 mg of an antithrombotic are mixed with 99.5 g of Vaseline under aseptic conditions.

### EXAMPLE E: TABLETS

A mixture of 1 kg of an active ingredient of the formula I, 1 kg of an antithrombotic, 4 kg of lactose, 1.2 kg of potato starch, 0.2 kg of talc and 0.1 kg of magnesium stearate is pressed to give tablets in a conventional manner in such a way that each tablet contains 10 mg of each active ingredient.

### EXAMPLE F: COATED TABLETS

Tablets are pressed analogously to Example E and subsequently coated in a conventional manner with a coating of sucrose, potato starch, talc, tragacanth and dye.

### EXAMPLE G: CAPSULES

2 kg of an active ingredient of the formula I and 2 kg of an antithrombotic are introduced into hard gelatine capsules in a conventional manner in such a way that each capsule contains 20 mg of each active ingredient.

### EXAMPLE H: AMPOULES

A solution of 1 kg of an active ingredient of the formula I and 1 kg of an antithrombotic in 60 l of bidistilled water is sterile filtered, transferred into ampoules, lyophilised under sterile conditions and sealed under sterile conditions. Each ampoule contains 10 mg of each active ingredient.

### EXAMPLE I: INHALATION SPRAY

14 g of an active ingredient of the formula I and 14 g of an antithrombotic are dissolved in 10 l of isotonic NaCl solution, and the solution is transferred into commercially available spray containers with a pump mechanism. The solution can be sprayed into the mouth or nose. One spray shot (about 0.1 ml) corresponds to a dose of about 0.14 mg of each active ingredient.

### EXAMPLE A′: INJECTION VIALS

A solution of 100 g of an active ingredient of the formula I, 100 g of the calcium antagonist and 5 g of disodium hydrogenphosphate in 3 l of bidistilled water is adjusted to pH 6.5 using 2N hydrochloric acid, sterile filtered, transferred into injection vials, lyophilised under sterile conditions and sealed under sterile conditions. Each injection vial contains 5 mg of each active ingredient.

### EXAMPLE B′: SUPPOSITORIES

A mixture of 20 g of an active ingredient of the formula I, 20 g of a calcium antagonist with 100 g of soya lecithin and 1400 g of cocoa butter is melted, poured into moulds and allowed to cool. Each suppository contains 20 mg of each active ingredient.

### EXAMPLE C′: SOLUTION

A solution is prepared from 1 g of an active ingredient of the formula I, 1 g of a calcium antagonist, 9.38 g of NaH₂PO₄.2H₂O, 28.48 g of Na₂HPO₄.12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE D′: OINTMENT 500 mg of an active ingredient of the formula I and 500 mg of a calcium antagonist are mixed with 99.5 g of Vaseline under aseptic conditions.

### EXAMPLE E′: TABLETS

A mixture of 1 kg of an active ingredient of the formula I, 1 kg of a calcium antagonist, 4 kg of lactose, 1.2 kg of potato starch, 0.2 kg of talc and 0.1 kg of magnesium stearate is pressed to give tablets in a conventional manner in such a way that each tablet contains 10 mg of each active ingredient.

### EXAMPLE F′: COATED tabl ts

Tablets are pressed analogously to Example E and subsequently coated in a conventional manner with a coating of sucrose, potato starch, talc, tragacanth and dye.

### EXAMPLE G′: CAPSULES

2 kg of an active ingredient of the formula I and 2 kg of a calcium antagonist are introduced into hard gelatine capsules in a conventional manner in such a way that each capsule contains 20 mg of each active ingredient.

### EXAMPLE H′: AMPOULES

A solution of 1 kg of an active ingredient of the formula I and 1 kg of a calcium antagonist in 60 l of bidistilled water is sterile filtered, transferred into ampoules, lyophilised under sterile conditions and sealed under sterile conditions. Each ampoule contains 10 mg of each active ingredient.

### EXAMPLE I′: INHALATION SPRAY

14 g of an active ingredient of the formula I and 14 g of a calcium antagonist are dissolved in 10 l of isotonic NaCl solution, and the solution is transferred into commercially available spray containers with a pump mechanism. The solution can be sprayed into the mouth or nose. One spray shot (about 0.1 ml) corresponds to a dose of about 0.14 mg of each active ingredient.

### EXAMPLE A″: INJECTION VIALS

A solution of 100 g of an active ingredient of the formula I, 100 g of the prostaglandin or prostaglandin derivative and 5 g of disodium hydrogen-phosphate in 3 l of bidistilled water is adjusted to pH 6.5 using 2N hydrochloric acid, sterile filtered, transferred into injection vials, lyophilised under sterile conditions and sealed under sterile conditions. Each injection vial contains 5 mg of each active ingredient.

### EXAMPLE B″: SUPPOSITORI s

A mixture of 20 g of an active ingredient of the formula I, 20 g of a prostaglandin or prostaglandin derivative with 100 g of soya lecithin and 1400 g of cocoa butter is melted, poured into moulds and allowed to cool. Each suppository contains 20 mg of each active ingredient.

### EXAMPLE C″: SOLUTION

A solution is prepared from 1 g of an active ingredient of the formula I, 1 g of a prostaglandin or prostaglandin derivative, 9.38 g of NaH₂PO₄.2H₂O, 28.48 g of Na₂HPO₄.12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE D″: OINTMENT

500 mg of an active ingredient of the formula I and 500 mg of a prostaglandin or prostaglandin derivative are mixed with 99.5 g of Vaseline under aseptic conditions.

### EXAMPLE E″: TABLETS

A mixture of 1 kg of an active ingredient of the formula I, 1 kg of a prostaglandin or prostaglandin derivative, 4 kg of lactose, 1.2 kg of potato starch, 0.2 kg of talc and 0.1 kg of magnesium stearate is pressed to give tablets in a conventional manner in such a way that each tablet contains 10 mg of each active ingredient.

### EXAMPLE F″: COATED TABLETS

Tablets are pressed analogously to Example E and subsequently coated in a conventional manner with a coating of sucrose, potato starch, talc, tragacanth and dye.

### EXAMPL G″: CAPSULES

2 kg of an active ingredient of the formula I and 2 kg of a prostaglandin or prostaglandin derivative are introduced into hard gelatine capsules in a conventional manner in such a way that each capsule contains 20 mg of each active ingredient.

### EXAMPLE H″: AMPOULES

A solution of 1 kg of an active ingredient of the formula I and 1 kg of a prostaglandin or prostaglandin derivative in 60 l of bidistilled water is sterile filtered, transferred into ampoules, lyophilised under sterile conditions and sealed under sterile conditions. Each ampoule contains 10 mg of each active ingredient.

### EXAMPLE I″: INHALATION SPRAY

14 g of an active ingredient of the formula I and 14 g of a prostaglandin or prostaglandin derivative are dissolved in 10 l of isotonic NaCl solution, and the solution is transferred into commercially available spray containers with a pump mechanism. The solution can be sprayed into the mouth or nose. One spray shot (about 0.1 ml) corresponds to a dose of about 0.14-mg of each active ingredient.

1. Pharmaceutical formulation comprising at least one phosphodiesterase V inhibitor and/or physiologically acceptable salts and/or solvates thereof and at least one antithrombotic.

2. Pharmaceutical formulation comprising at least one compound of the formula I 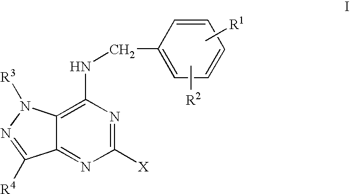
in which
R¹ and R² are each, independently of one another, H, A, OH, OA or Hal,
R¹ and R² together are alternatively alkylene having 3-5 carbon atoms, —O—CH₂—CH₂—, —CH₂—O—CH₂—, —O—CH₂—O— or —O—CH₂—CH₂—O—,
R³ and R⁴ are each, independently of one another, H or A,
X is R⁵, R⁶ or R⁷ which is monosubstituted by R⁸,
R⁵ is linear or branched alkylene having 1-10 carbon atoms, in which one or two CH₂ groups may be replaced by —CH═CH— groups, O, S or SO,
R⁶ is cycloalkyl or cycloalkylalkylene having 5-12 carbon atoms,
R⁷ is phenyl or phenylmethyl,
R⁸ is COOH, COOA, CONH₂, CONHA, CON(A)₂ or CN,
A is alkyl having from 1 to 6 carbon atoms, and
Hal is F, Cl, Br or I,
and/or physiologically acceptable salts and/or solvates thereof and
a) at least one antithrombotic or
b) at least one calcium antagonist or
c) at least one prostaglandin or prostaglandin derivative.

3. Pharmaceutical formulation according to claim 2, comprising at least one compound of the formula I 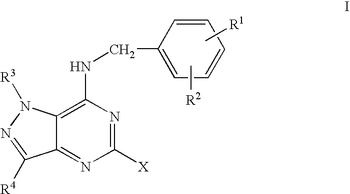
in which
R¹ and R² are each, independently of one another, H, A, OH, OA or Hal,
R¹ and R² together are alternatively alkylene having 3-5 carbon atoms, —O—CH₂—CH₂—, —CH₂—O—CH₂—, —O—CH₂—O— or —O—CH₂—CH₂—O—,
R³ and R⁴ are each, independently of one another, H or A,
X is R⁵, R⁶ or R⁷ which is monosubstituted by R⁸,
R⁵ is linear or branched alkylene having 1-10 carbon atoms, in which one or two CH₂ groups may be replaced by —CH═CH— groups, O, S or SO,
R⁶ is cycloalkyl or cycloalkylalkylene having 5-12 carbon atoms,
R⁷ is phenyl or phenylmethyl,
R⁸ is COOH, COOA, CONH₂, CONHA, CON(A)₂ or CN,
A is alkyl having from 1 to 6 carbon atoms, and
Hal is F, Cl, Br or I,
and/or physiologically acceptable salts and/or solvates thereof and at least one antithrombotic.

4. Pharmaceutical formulation according to claim 3, comprising at least one compound of the formula I according to claim 3 in which
X is R⁵, phenyl or phenylmethyl, each of which is substituted by COOH, COOA, CONH₂, CONA₂, CONHA or CN;
and/or physiologically acceptable salts and/or solvates thereof and at least one antithrombotic.

5. Pharmaceutical formulation according to claim 3, comprising at least one compound of the formula I according to claim 3 in which
R¹ and R² together are alkylene having 3-5 carbon atoms, —O—CH₂—CH₂—, —O—CH₂—O— or —O—CH₂—CH₂—O—,
X is R⁵, phenyl or phenylmethyl, each of which is substituted by COOH, COOA, CONH₂, CONA₂, CONHA or CN;
and/or physiologically acceptable salts and/or solvates thereof and at least one antithrombotic.

6. Pharmaceutical formulation according to claim 3, comprising at least one compound of the formula I according to claim 3 in which
R¹ and R² are each, independently of one another, H, A, OH, OA or Hal,
R¹ and R² together are alternatively alkylene having 3-5 carbon atoms, —O—CH₂—CH₂—, —O—CH₂—O— or —O—CH₂—CH₂—O—,
X is R⁵, phenyl or phenylmethyl, each of which is substituted by COOH, COOA, CONH₂, CONA₂, CONHA or CN;
and/or physiologically acceptable salts and/or solvates thereof and at least one antithrombotic.

7. Pharmaceutical formulation according to claim 3, comprising at least one compound of the formula I according to claim 3 in which
R¹ and R² are each, independently of one another, H, A, OH, OA or Hal,
R¹ and R² together are alternatively alkylene having 3-5 carbon atoms, —O—CH₂—CH₂—, —O—CH₂—O— or —O—CH₂—CH₂—O—,
X is alkylene having 2-5 carbon atoms, cyclohexyl, phenyl or phenylmethyl, each of which is monosubstituted by R⁸,
R³ is alkyl having 1-6 carbon atoms,
R⁴ is alkyl having 1-6 carbon atoms,
R⁸ is COOH or COOA,
A is alkyl having from 1 to 6 carbon atoms,
Hal is F, Cl, Br or I;
and/or physiologically acceptable salts and/or solvates thereof and at least one antithrombotic.

8. Pharmaceutical formulation according to claim 3, comprising at least one compound of the formula I according to claim 3 in which
R¹ and R² are each, independently of one another, H, A, OH, OA or Hal,
R¹ and R² together are alternatively alkylene having 3-5 carbon atoms, —O—CH₂—CH₂—, —O—CH₂—O— or —O—CH₂—CH₂—O—,
R³ is alkyl having 1-6 carbon atoms,
R⁴ is alkyl having 1-6 carbon atoms,
X is —(CH₂)₂₋₅—R⁸, in which one CH₂ group may be replaced by O, or is 4-R⁸-cyclohexyl, 4-R⁸-phenyl or 4-(R⁸-methyl)phenyl,
R⁸ is COOH or COOA;
and/or physiologically acceptable salts and/or solvates thereof and at least one antithrombotic.

9. Pharmaceutical formulation according to claim 3, comprising at least one compound of the formula I according to claim 3 selected from the group consisting of
(a) 5-[7-(3-chloro-4-methoxybenzylamino-1-methyl-3-propyl-1 H-pyrazolo[4,3-d]pyrimidin-5-yl]pentanoic acid;
(b) 4-[7-(3-chloro-4-methoxybenzylamino)1-methyl-3-propyl-1H-pyrazolo[4,3-d]pyrimidin-5-yl]benzoic acid;
(c) 4-[7-(3,4-methylenedioxybenzylamino)1-methyl-3-propyl-1H-pyrazolo[4,3-d]pyrimidin-5-yl]butyric acid;
(d) 5-[7-(benzylamino)-1-methyl-3-propyl-1H-pyrazolo[4,3-d]-pyrimidin-5-yl]pentanoic acid;
(e) [7-(3-chloro-4-methoxybenzylamino)-1-methyl-3-propyl-1H-pyrazolo[4,3-d]pyrimidin-5-ylmethoxy]acetic acid and/or physiologically acceptable salts and/or solvates thereof and at least one antithrombotic.

10. Pharmaceutical formulation according to claim 9, comprising at least [7-(3-chloro-4-methoxybenzylamino)-1-methyl-3-propyl-1H-pyrazolo-[4,3-d]pyrimidin-5-ylmethoxy]acetic acid, ethanolamine salt, and at least one antithrombotic.

11. Pharmaceutical formulation according to claims 1 to 10, in which the antithrombotic is selected from the group consisting of vitamin K antagonists, heparin compounds, thrombocyte aggregation inhibitors, enzymes, factor Xa inhibitors, factor VIIa inhibitors and other antithrombotic agents.

12. Pharmaceutical formulation according to claim 11, where the vitamin K antagonists are selected from the group consisting of dicoumarol, phenindione, warfarin, phenprocoumon, acenocoumarol, ethyl biscoumacetate, clorindione, diphenadione and tioclomarol.

13. Pharmaceutical formulation according to claim 11, where the heparin compounds are selected from the group consisting of heparin, antithrombin III, dalteparin, enoxaparin, nadroparin, parnaparin, reviparin, danaparoid, tinzaparin and sulodexide.

14. Pharmaceutical formulation according to claim 11, where the thrombocyte aggregation inhibitors are selected from the group consisting of ditazole, cloricromen, picotamide, clopidogrel, ticlopidine, acetylsalicylic acid, dipyridamole, calcium carbassalate, epoprostenol, indobufen, iloprost, abciximab, tirofiban, aloxiprin and intrifiban.

15. Pharmaceutical formulation according to claim 11, where the enzymes are selected from the group consisting of streptokinase, alteplase, anistreplase, urokinase, fibrinolysin, brinase, reteplase and saruplase.

16. Pharmaceutical formulation according to claim 11, where other antithrombotic agents are selected from the group consisting of defibrotide, desirudin and lepirudin.

17. Pharmaceutical formulation according to claims 1-10, where the antithrombotic is selected from the group consisting of blood platelet glycoprotein receptor (IIb/IIIa) antagonists.

18. Pharmaceutical formulation according to claim 2, comprising at least one compound of the formula I 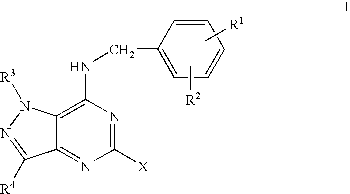
in which
R¹ and R² are each, independently of one another, H, A, OH, OA or Hal,
R¹ and R² together are alternatively alkylene having 3-5 carbon atoms, —O—CH₂—CH₂—, —CH₂—O—CH₂—, —O—CH₂—O— or —O—CH₂—CH₂—O—,
R³ and R⁴ are each, independently of one another, H or A,
X is R⁵, R⁶ or R⁷, each of which is monosubstituted by R⁸,
R⁵ is linear or branched alkylene having 1-10 carbon atoms, in which one or two CH₂ groups may be replaced by —CH═CH— groups, O, S or SO,
R⁶ is cycloalkyl or cycloalkylalkylene having 5-12 carbon atoms,
R⁷ is phenyl or phenylmethyl,
R⁸ is COOH, COOA, CONH₂, CONHA, CON(A)₂ or CN,
A is alkyl having from 1 to 6 carbon atoms, and
Hal is F, Cl, Br or I,
and/or physiologically acceptable salts and/or solvates thereof and at least one calcium antagonist.

19. Pharmaceutical formulation according to claim 18, comprising at least one compound of the formula I according to claim 18 in which
X is R⁵, phenyl or phenylmethyl, each of which is substituted by COOH, COOA, CONH₂, CONA₂, CONHA or CN;
and/or physiologically acceptable salts and/or solvates thereof and at least one calcium antagonist.

20. Pharmaceutical formulation according to claim 18, comprising at least one compound of the formula I according to claim 18 in which
R¹ and R² together are alkylene having 3-5 carbon atoms, —O—CH₂—CH₂—, —O—CH₂—O— or —O—CH₂—CH₂—O—,
X is R⁵, phenyl or phenylmethyl, each of which is substituted by COOH, COOA, CONH₂, CONA₂, CONHA or CN;
and/or physiologically acceptable salts and/or solvates thereof and at least one calcium antagonist.

21. Pharmaceutical formulation according to claim 18, comprising at least one compound of the formula I according to claim 18 in which
R¹ and R² are each, independently of one another, H, A, OH, OA or Hal,
R¹ and R² together are alternatively alkylene having 3-5 carbon atoms, —O—CH₂—CH₂—, —O—CH₂—O— or —O—CH₂—CH₂—O—,
X is R⁵, phenyl or phenylmethyl, each of which is substituted by COOH, COOA, CONH₂, CONA₂, CONHA or CN;
and/or physiologically acceptable salts and/or solvates thereof and at least one calcium antagonist.

22. Pharmaceutical formulation according to claim 18, comprising at least one compound of the formula I according to claim 18 in which
R¹ and R² are each, independently of one another, H, A, OH, OA or Hal,
R¹ and R² together are alternatively alkylene having 3-5 carbon atoms, —O—CH₂—CH₂—, —O—CH₂—O— or —O—CH₂—CH₂—O—,
X is alkylene having 2-5 carbon atoms, cyclohexyl, phenyl or phenylmethyl, each of which is monosubstituted by R⁸,
R³ is alkyl having 1-6 carbon atoms,
R⁴ is alkyl having 1-6 carbon atoms,
R⁸ is COOH or COOA,
A is alkyl having from 1 to 6 carbon atoms,
Hal is F, Cl, Br or I;
and/or physiologically acceptable salts and/or solvates thereof and at least one calcium antagonist.

23. Pharmaceutical formulation according to claim 18, comprising at least one compound of the formula I according to claim 18 in which
R¹ and R² are each, independently of one another, H, A, OH, OA or Hal,
R¹ and R² together are alternatively alkylene having 3-5 carbon atoms, —O—CH₂—CH₂—, —O—CH₂—O— or —O—CH₂—CH₂—O—,
R³ is alkyl having 1-6 carbon atoms,
R⁴ is alkyl having 1-6 carbon atoms,
x is —(CH₂)₂₋₅—R⁸, in which one CH₂ group may be replaced by 0, or is 4-R⁸-cyclohexyl, 4-R⁸-phenyl or 4-(R⁸-methyl)phenyl,
R⁸ is COOH or COOA;
and/or physiologically acceptable salts and/or solvates thereof and at least one calcium antagonist.

24. Pharmaceutical formulation according to claim 18, comprising at least one compound of the formula I according to claim 18 selected from the group consisting of
(a) 5-[7-(3-chloro-4-methoxybenzylamino)-1-methyl-3-propyl-1H-pyrazolo[4,3-d]pyrimidin-5-yl]pentanoic acid;
(b) 4-[7-(3-chloro-4-methoxybenzylamino)-1-methyl-3-propyl-1H-pyrazolo[4,3-d]pyrimidin-5-yl]benzoic acid;
(c) 4-[7-(3,4-methylenedioxybenzylamino)-1-methyl-3-propyl-1 H-pyrazolo[4,3-d]pyrimidin-5-yl]butyric acid;
(d) 5-[7-(benzylamino)-1-methyl-3-propyl-1H-pyrazolo[4,3-d]-pyrimidin-5-yl]pentanoic acid;
(e) [7-(3-chloro-4-methoxybenzylamino)-1-methyl-3-propyl-1H-pyrazolo[4,3-d]pyrimidin-5-ylmethoxy]acetic acid and/or physiologically acceptable salts and/or solvates thereof and at least one calcium antagonist.

25. Pharmaceutical formulation according to claim 24, comprising at least [7-(3-chloro-4-methoxybenzylamino)-1-methyl-3-propyl-1H-pyrazolo[4,3-d]pyrimidin-5-ylmethoxy]acetic acid, ethanolamine salt, and at least one calcium antagonist.

26. Pharmaceutical formulation according to claims 2 and 18 to 25, in which the calcium antagonist is selected from the group consisting of selective and non-selective calcium antagonists.

27. Pharmaceutical formulation according to claim 26, in which the selective calcium antagonists are selected from the group consisting of dihydropyridine derivatives, phenylalkylamine derivatives, benzothiazepine derivatives and other selective calcium antagonists.

28. Pharmaceutical formulation according to claim 27, in which the dihydropyridine derivatives are selected from the group consisting of amlodipine, felodipine, isradipine, nicardipine, nifedipine, nimodipine, nisoldipine, nitrendipine, lacidipine, nilvadipine, manidipine, bamidipine and lercanidipine.

29. Pharmaceutical formulation according to claim 27, in which the phenylalkylamine derivatives are selected from the group consisting of verapamil and gallopamil.

30. Pharmaceutical formulation according to claim 27, in which the benzothiazepine derivative is diltiazem.

31. Pharmaceutical formulation according to claim 27, in which the other selective calcium antagonist is mibefradil.

32. Pharmaceutical formulation according to claim 26, in which the nonselective calcium antagonists are selected from the group consisting of fendiline, bepridil, lidoflazine and perhexiline.

33. Pharmaceutical formulation according to claim 2, comprising at least one compound of the formula I 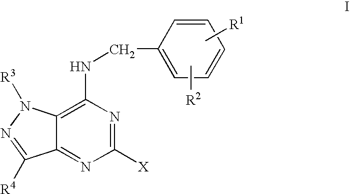
in which
R¹ and R² are each, independently of one another, H, A, OH, OA or Hal,
R¹ and R² together are alternatively alkylene having 3-5 carbon atoms, —O—CH₂—CH₂—, —CH₂—O—CH₂—, —O—CH₂—O— or —O—CH₂—CH₂—O—,
R³ and R⁴ are each, independently of one another, H or A,
X is R⁵, R⁶ or R⁷, each of which is monosubstituted by R⁸,
R⁵ is linear or branched alkylene having 1-10 carbon atoms, in which one or two CH₂ groups may be replaced by —CH═CH— groups, O, S or SO,
R⁶ is cycloalkyl or cycloalkylalkylene having 5-12 carbon atoms,
R⁷ is phenyl or phenylmethyl,
R⁸ is COOH, COOA, CONH₂, CONHA, CON(A)₂ or CN,
A is alkyl having from 1 to 6 carbon atoms, and
Hal is F, Cl, Br or I,
and/or physiologically acceptable salts and/or solvates thereof and at least one prostaglandin or prostaglandin derivative.

34. Pharmaceutical formulation according to claim 33, comprising at least one compound of the formula I according to claim 33 in which
X is R⁵, phenyl or phenylmethyl, each of which is substituted by COOH, COOA, CONH₂, CONA₂, CONHA or CN;
and/or physiologically acceptable salts and/or solvates thereof and at least one prostaglandin or prostaglandin derivative.

35. Pharmaceutical formulation according to claim 33, comprising at least one compound of the formula I according to claim 33 in which
R¹ and R² together are alkylene having 3-5 carbon atoms, —O—CH₂—CH₂—, —O—CH₂—O— or —O—CH₂—CH₂—O—,
X is R⁵, phenyl or phenylmethyl, each of which is substituted by COOH, COOA, CONH₂, CONA₂, CONHA or CN;
and/or physiologically acceptable salts and/or solvates thereof and at least one prostaglandin or prostaglandin derivative.

36. Pharmaceutical formulation according to claim 33, comprising at least one compound of the formula I according to claim 33 in which
R¹ and R² are each, independently of one another, H, A, OH, OA or Hal,
R¹ and R² together are alternatively alkylene having 3-5 carbon atoms, —O—CH₂—CH₂—, —O—CH₂—O— or —O—CH₂—CH₂—O—,
X is R⁵, phenyl or phenylmethyl, each of which is substituted by COOH, COOA, CONH₂, CONA₂, CONHA or CN;
and/or physiologically acceptable salts and/or solvates thereof and at least one prostaglandin or prostaglandin derivative.

37. Pharmaceutical formulation according to claim 33, comprising at least one compound of the formula I according to claim 33 in which
R¹ and R² are each, independently of one another, H, A, OH, OA or Hal,
R¹ and R² together are alternatively alkylene having 3-5 carbon atoms, —O—CH₂—CH₂—, —O—CH₂—O— or —O—CH₂—CH₂—O—,
X is alkylene having 2-5 carbon atoms, cyclohexyl, phenyl or phenylmethyl, each of which is monosubstituted by R⁸,
R³ is alkyl having 1-6 carbon atoms,
R⁴ is alkyl having 1-6 carbon atoms, R⁸ is COOH or COOA,
A is alkyl having from 1 to 6 carbon atoms,
Hal is F, Cl, Br or I;
and/or physiologically acceptable salts and/or solvates thereof and at least one prostaglandin or prostaglandin derivative.

38. Pharmaceutical formulation according to claim 33, comprising at least one compound of the formula I according to claim 33 in which
R¹ and R² are each, independently of one another, H, A, OH, OA or Hal,
R¹ and R² together are alternatively alkylene having 3-5 carbon atoms, —O—CH₂—CH₂—, —O—CH₂—O— or —O—CH₂—CH₂—O—,
R³ is alkyl having 1-6 carbon atoms,
R⁴ is alkyl having 1-6 carbon atoms,
X is —(CH₂)₂₋₅—R⁸, in which one CH₂ group may be replaced by O, or is 4-R⁸-cyclohexyl, 4-R⁸-phenyl or 4-(R⁸-methyl)phenyl,
R⁸ is COOH or COOA;
and/or physiologically acceptable salts and/or solvates thereof and at least one prostaglandin or prostaglandin derivative.

39. Pharmaceutical formulation according to claim 33, comprising at least one compound of the formula I according to claim 33 selected from the group consisting of
(a) 5-[7-(3-chloro-4-methoxybenzylamino)-1-methyl-3-propyl-1H-pyrazolo[4,3-d]pyrimidin-5-yl]pentanoic acid;
(b) 4-[7-(3-chloro-4-methoxybenzylamino)-1-methyl-3-propyl-1H-pyrazolo[4,3-d]pyrimidin-5-yl]benzoic acid;
(c) 4-[7-(3,4-methylenedioxybenzylamino)-1-methyl-3-propyl-1H-pyrazolo[4,3-d]pyrimidin-5-yl]butyric acid;
(d) 5-[7-(benzylamino)-1-methyl-3-propyl-1H-pyrazolo[4,3-d]-pyrimidin-5-yl]pentanoic acid;
(e) [7-(3-chloro-4-methoxybenzylamino)-1-methyl-3-propyl-1H-pyrazolo[4,3-d]pyrimidin-5-ylmethoxy]acetic acid
and/or physiologically acceptable salts and/or solvates thereof and at least one prostaglandin or prostaglandin derivative.

40. Pharmaceutical formulation according to claim 39, comprising at least [7-(3-chloro-4-methoxybenzylamino)-1-methyl-3-propyl-1H-pyrazolo[4,3-d]pyrimidin-5-ylmethoxy]acetic acid, ethanolamine salt, and at least one prostaglandin or prostaglandin derivative.

41. Pharmaceutical formulation according to claims 2 and 33 to 40, in which the prostaglandin or prostaglandin derivative is selected from the group consisting of alprostadil (PGE₁), dinoprost (PGF₂), dinoprostone (PGE₂), epoprostenol sodium (PGI₂; prostacyclin sodium), gemeprost, iloprost, latanoprost, misoprostol, sulprostone, carboprost, thromethamin, dinoprost thromethamin, lipoprost, metenoprost and tiaprost.

42. Pharmaceutical formulation according to claim 41, in which the prostaglandin is PGE₁ or prostacyclin.

43. Pharmaceutical formulation according to claim 41, in which the prostaglandin is prostacyclin.

44. Pharmaceutical formation according to one of the preceding claims, comprising one or more excipients and/or assistants.

45. Use of a pharmaceutical preparation according to one of claims 1 to 44 for the preparation of a medicament for the treatment of angina, high blood pressure, pulmonary hypertension, congestive heart failure (CHF), chronic obstructive pulmonary disease (COPD), cor pulmonale, dextrocardiac insufficiency, atherosclerosis, conditions of reduced patency of heart vessels, peripheral vascular diseases, strokes, bronchitis, allergic asthma, chronic asthma, allergic rhinitis, glaucoma, irritable bowel syndrome, tumours, renal insufficiency, liver cirrhosis and for the treatment of female sexual disorders.

46. Use according to claim 45 for the preparation of a medicament for the treatment of pulmonary hypertension, congestive heart failure (CHF), chronic obstructive pulmonary disease (COPD), cor pulmonale and/or dextrocardiac insufficiency.

47. Set (kit) consisting of separate packs of
(a) an effective amount [7-(3-chloro-4-methoxybenzylamino)-1-methyl-3-propyl-1H-pyrazolo[4,3-d]pyrimidin-5-ylmethoxy]acetic acid, ethanolamine salt, and
(b) an effective amount of an antithrombotic.

48. Use of [7-(3-chloro-4-methoxybenzylamino)-1-methyl-3-propyl-1H-pyrazolo[4,3-d]pyrimidin-5-ylmethoxy]acetic acid, ethanolamine salt, for the preparation of a medicament for the treatment of pulmonary hypertension, congestive heart failure (CHF), chronic obstructive pulmonary disease (COPD), cor pulmonale and/or dextrocardiac insufficiency.

49. Set (kit) consisting of separate packs of
(a) an effective amount of [7-(3-chloro-4-methoxybenzylamino)-1-methyl-3-propyl-1H-pyrazolo[4,3-d]pyrimidin-5-ylmethoxy]acetic acid, ethanolamine salt, and
(b) an effective amount of a calcium antagonist.

50. Set (kit) consisting of separate packs of
(a) an effective amount of [7-(3-chloro-4-methoxybenzylamino)-1-methyl-3-propyl-1H-pyrazolo[4,3-d]pyrimidin-5-ylmethoxy]acetic acid, ethanolamine salt, and
(b) an effective amount of a prostaglandin or prostaglandin derivative.

51. Use of a pharmaceutical preparation comprising at least one phosphodiesterase V inhibitor and at least one prostaglandin or prostaglandin derivative for the preparation of a medicament for the oral treatment of pulmonary hypertension, congestive heart failure (CHF), chronic obstructive pulmonary disease (COPD), cor pulmonale and/or dextrocardiac insufficiency.
